(19) United States
(12) Patent Application Publication
(10) Pub. No.: US 2021/0315639 A1
(43) Pub. Date: Oct. 14, 2021

(54) Current Analysis for Patient Customized Irreversible Electroporation Treatment Planning

(71) Applicant: ANGIODYNAMICS, INC., Latham, NY (US)

(72) Inventors: Navid MANUCHERHABADI, Marlborough, MA (US); HAYLEY FORREST, Framingham, MA (US)

(73) Assignee: ANGIODYNAMICS, INC., Latham, NY (US)

(21) Appl. No.: 17/229,632

(22) Filed: Apr. 13, 2021

Related U.S. Application Data
(60) Provisional application No. 63/009,040, filed on Apr. 13, 2020.
(60) Provisional application No. 63/031,282, filed on May 28, 2020.

(51) Int. Cl. A61B 34/10; A61B 18/12; G16H 20/30; G06N 20/00

(52) U.S. Cl. CPC A61B 34/10; A61B 18/1206; A61B 2018/00613; G06N 20/00; G16H 20/30

(57) ABSTRACT

Provided are devices and techniques to compare current across treatment plans as well as between various clinician and/or clinic protocols. Current from an ablation therapy treatment can be measured and normalized to compare the normalized current between ablation therapy treatments. An efficacy of a treatment, a real-time display of treatment progression, a completion of a treatment, and/or effective treatment planning can be determined based on a change (or a potential change) in the normalized current. Also provided is a database of treatment results including indications of normalized current from the treatments.

### CROSS-REFERENCE TO RELATED APPLICATIONS DD

This application claims priority to U.S. provisional application No. 63/009,040, filed on Apr. 13, 2020, and U.S. provisional application No. 63/031,282, filed on May 28, 2020, both of which are incorporated herein by reference in their entirety.

### BACKGROUND

Irreversible electroporation (IRE) and high-frequency IRE (H-FIRE) are interventional oncology techniques, which may trigger a range of different cell death mechanisms thereby ablating the target tissue. IRE and H-FIRE are often used to ablate tumors in regions where precision and conservation of the critical structures such as extracellular matrix, blood vessel and nerves are of importance.

Examples of available techniques to analyze the extent of tissue ablation (including thermal and/or non-thermal modalities) are in situ dynamic electrical conductivity measurements, fluroptic or thermocouple based local temperature measurements, temporal and spatial MRI guided temperature mapping, ultrasound, and computer tomography. However, these tools are often invasive, expensive, and need data post processing, which limits their usage for clinical applications. Furthermore, a number of these tools may interfere with the electrical field used to cause cell death, and consequently the ablation volume, in IRE or H-FIRE procedures. As such, these tools are often impractical to use to determine the extent of tissue ablation for IRE and H-FIRE procedures.

### BRIEF SUMMARY

The present disclosure provides techniques to compare current output across treatment plans as well as between various clinician and/or clinic protocols. In general, the present disclosure provides methods and devices to normalize the current response from an IRE or H-FIRE treatment and compare normalized current between treatments. In some examples, the present disclosure provides methods and devices to determine an intensity of an IRE or H-FIRE treatment, or plan for an IRE or H-FIRE treatment based on a rate of change of the normalized current or a percentage change or rise in the normalized current. This provides significant advantages over conventional techniques clinicians use to identify the completion of a treatment. Furthermore, the present disclosure overcomes difficulties associated with comparing different treatments, which have prevented sharing of data between clinicians and clinics. As discussed herein, the system may also be able to detect the transition between primarily IRE and primarily non-IRE treatment zones.

As such, the present disclosure can be implemented by clinics to form databases or “banks” of tissue specific treatments representing treatments from multiple clinicians and even multiple clinics. This database can include information about normalized current from these procedures, which can be utilized by clinicians or researchers for treatment planning. Furthermore, ablation therapy devices could compare normalized current associated with an in-progress therapy procedure to normalized current from the database to provide intraprocedural information regarding the progress, extent and/or effectiveness of the therapy procedure or to suggest continued treatment procedure parameters (e.g., additional voltage pulses, or the like).

A benefit to the present disclosure, or said differently, to representing current using equations given herein and normalizing the current to compare current across treatments is that the present disclosure can be used to simplify the impact of the numerous variables (including intrinsic and extrinsic properties as described below in more detail) associated with ablation volume in IRE and H-FIRE treatments. A number of additional benefits can be realized from the present disclosure such as, for example: predicting the current response after an increase or decrease in the applied voltage before or during a procedure; predicting the current response after a physical change is made during the procedure such as pullback, probe repositioning, or electrode exposure change; avoiding overcurrent, especially at higher voltages, by using typical normalized current increases over a certain number of pulses; accessing and compare data between clinicians or clinics; generating a bank of results for various tissue types to compare treatments with; directly comparing current response between voltages by removing the offsets to better predict the effectiveness of a specific protocol; predicting a zone of treatment given the normalized current and a derived rate of change; and estimating the dynamic electrical conductivity of tissue.

These and other examples are described in greater detail below. In the following description, numerous specific details such as processor and system configurations are set forth in order to provide a more thorough understanding of the described embodiments. However, the described embodiments may be practiced without such specific details. Additionally, some well-known structures (e.g., circuits, specific treatment protocols, and the like) have not been shown in detail, to avoid unnecessarily obscuring the described embodiments.

In one embodiment, an ablation therapy device comprises a generator, a sensor, a processor, and a memory, the processor coupled to the generator, the sensor, and the memory; the generator to operatively couple to a plurality of electrodes, and the generator to generate a plurality of electrical pulses to be applied through the electrodes to a target tissue; the sensor arranged to measure a current produced responsive to application of the plurality of electrical pulses to the target tissue; and memory storing instructions, which when executed by the processor cause the processor to receive from the sensor, an indication of the current; and normalize the current.

The instructions, when executed by the processor cause the processor to determine whether a difference between the normalized current for a first electrical pulse of the plurality of electrical pulses and the normalized current for a second electrical pulse of the plurality of electrical pulses is greater than a threshold value; and generate a control signal comprising an indication to pause generation of the plurality of electrical pulses based on a determination that the difference between the normalized current for the first electrical pulse of the plurality of electrical pulses and the normalized current for the second electrical pulse of the plurality of electrical pulses is greater than the threshold value.

The device further comprising a display unit coupled to the processor; and the instructions, when executed by the processor cause the processor to generate a first graphical information element comprising an indication of a plot of the normalized current; generate a second graphical information element comprising an indication of a query of whether to continue generation of the plurality of electrical pulses; and send the first graphical information element and the second graphical information element to the display unit to cause the display unit to display the plot and the query.

Wherein the sensor comprises a voltage sensor, a current sensor, or a voltage sensor and a current sensor; and wherein the normalized current comprises extrinsic factors and intrinsic factors.

Wherein the plurality of electrical pulses are sufficient to substantially reversibly electroporate cells within the target tissue, irreversibly electroporate cells within the target tissue, thermally ablate cells within the target tissue, and/or result in electrolysis of cells within the target tissue.

The instructions, when executed by the processor cause the processor to normalize the first current data based in part on a standard deviation, mean, or coefficient of variation.

The instructions, when executed by the processor cause the processor to normalize the rate of change of the current.

Wherein the sensor arranged to measure a conductivity produced responsive to application of the plurality of electrical pulses to the target tissue; and wherein the memory storing instructions, which when executed by the processor cause the processor to receive from the sensor, an indication of the conductivity; and normalize the conductivity.

The instructions, when executed by the processor cause the processor to generate a control signal based on the normalized current; and send the control signal to the generator.

The device further comprising a memory storing a machine learning (ML) model and instructions, the instructions when executed by the processor cause the processor to execute the ML model to generate an inference of the normalized current based on the indication of the sensed current.

In one embodiment, an ablation device, comprising a voltage source to generate a plurality of electrical pulses to be applied to a target site, the voltage source to operatively couple to a plurality of electrodes; a sensor coupled to at least one of the plurality of electrodes, the sensor arranged to measure an electrical characteristic associated with application of the plurality of electrical pulses to the target tissue; a processor; and memory storing a machine learning (ML) model and instructions, the instructions when executed by the processor cause the processor to receive from the sensor, an indication of the characteristic; normalize the electrical characteristic; execute the ML model to generate an inference of the normalized electrical characteristic based on the indication of the electrical characteristic; and generate a graphical information element comprising the indication of the normalized electrical characteristic of the ablation therapy.

Wherein the sensor comprises a voltage sensor, a current sensor, or a voltage sensor and a current sensor and wherein the electrical characteristic comprises a current, a voltage, or a current and a voltage.

The instructions, when executed by the processor cause the processor to receive an indication of a type of the target tissue; and execute the ML model to generate the inference of the normalized electrical characteristic of the ablation therapy based on the indication of the normalized electrical characteristic and the type of the target tissue.

The instructions, when executed by the processor cause the processor to receive at least one of an indication of patient demographics or an indication of protocol parameters of the ablation therapy; and execute the ML model to generate the inference of the normalized electrical characteristic of the ablation therapy based on the indication of the normalized electrical characteristic, the type of the target tissue, and the at least one of the indications of patient demographics or the indication of protocol parameters of the ablation therapy.

Wherein the normalized electrical characteristic of the ablation therapy comprises one or more of normalized current, normalized conductivity, an ablation therapy zone, a rate of change in normalized current versus a quantity of the plurality of voltage pulses, or a rate of change in normalized conductivity versus the quantity of the plurality of voltage pulses.

The instructions, when executed by the processor cause the processor to receive an indication from the voltage source of a second plurality of voltage pulses to be applied to the target tissue via the plurality of electrodes as part of the ablation therapy; execute the ML model to generate an updated inference of an updated normalized electrical characteristic of the ablation therapy based on the electrical characteristic and the second plurality of voltage pulses; and generate a second graphical information element comprising the indication of the updated normalized electrical characteristic of the ablation therapy.

The instructions when executed by the processor cause the processor to execute the ML model to generate an inference of the normalized electrical characteristic and suggested protocol parameters of the ablation therapy; and generate the graphical information element comprising the indication of the normalized electrical characteristic of the ablation therapy and an indication of the suggested protocol parameters.

In one embodiment, a method, comprising receiving from a sensor, an indication of an electrical characteristic generated responsive to at least one electrical pulse applied to a target tissue by a plurality of electrodes operatively coupled to an ablation therapy device; normalizing the electrical characteristic; generating a control signal for the ablation therapy device based on the normalized electrical characteristic; and sending the control signal to the ablation therapy device.

Wherein the step of normalizing the electrical characteristic comprises both extrinsic factors and intrinsic factors.

Wherein the sensor comprises a voltage sensor, a current sensor, or a voltage sensor and a current sensor and wherein the electrical characteristic comprises a current, a voltage, or a current and a voltage; and further comprising the steps generating a first graphical information element comprising an indication of a plot of the normalized electrical characteristic; generating a second graphical information element comprising an indication of a query of whether to continue application of the plurality of electrical pulses; and displaying on a display device, based on the first graphical information element and the second graphical information element, the plot and the query.

In one embodiment, an ablation therapy device, comprising a voltage source to generate a plurality of voltage pulses to be applied to a target tissue via a plurality of probes as part of an ablation therapy; a sensor coupled to at least one of the plurality of probes, the sensor arranged to measure an extrinsic characteristic associated with application of the plurality of voltage pulses to the target tissue; a processor; and memory storing a machine learning (ML) model and instructions, the instructions when executed by the processor cause the processor to receive from the sensor, an indication of the extrinsic characteristic; execute the ML model to generate an inference of an intrinsic characteristic of the ablation therapy based on the indication of the extrinsic characteristic; and generate a graphical information element comprising the indication of the intrinsic characteristic of the ablation therapy.

The instructions, when executed by the processor cause the processor to receive an indication of a type of the target tissue; and execute the ML model to generate the inference of the intrinsic characteristic of the ablation therapy based on the indication of the extrinsic characteristic and the type of the target tissue.

The instructions, when executed by the processor cause the processor to receive at least one of an indication of patient demographics or an indication of protocol parameters of the ablation therapy; and execute the ML model to generate the inference of the intrinsic characteristic of the ablation therapy based on the indication of the extrinsic characteristic, the type of the target tissue, and the at least one of the indications of patient demographics or the indication of protocol parameters of the ablation therapy.

Wherein the intrinsic characteristic of the ablation therapy comprises one or more of normalized current, normalized conductivity, an ablation therapy zone, a rate of change in normalized current versus a quantity of the plurality of voltage pulses, or a rate of change in normalized conductivity versus the quantity of the plurality of voltage pulses.

The instructions, when executed by the processor cause the processor to receive an indication from the voltage source of a second plurality of voltage pulses to be applied to the target tissue via the plurality of probes as part of the ablation therapy; execute the ML model to generate an updated inference of an updated intrinsic characteristic of the ablation therapy based on the extrinsic characteristic and the second plurality of voltage pulses; and generate a second graphical information element comprising the indication of the updated intrinsic characteristic of the ablation therapy.

The instructions when executed by the processor cause the processor to execute the ML model to generate an inference of the intrinsic characteristic and suggested protocol parameters of the ablation therapy; and generate the graphical information element comprising the indication of the intrinsic characteristic of the ablation therapy and an indication of the suggested protocol parameters.

In one embodiment, An ablation therapy device system, comprising a database comprising data associated with a plurality of ablation therapy procedures, the data comprising patient demographic data, protocol parameter data, and post-procedure results data; an ablation therapy device, comprising a voltage source to generate a plurality of voltage pulses to be applied to a target tissue via a plurality of probes as part of an active ablation therapy procedure; a sensor coupled to at least one of the plurality of probes, the sensor arranged to measure an extrinsic characteristic associated with the active ablation therapy procedure; a processor; and memory storing instructions, which when executed by the processor cause the processor to receive from the voltage source, an indication of the plurality of voltage pulses; receive from the sensor, an indication of the extrinsic characteristic; add, to the database, an indication of the active ablation therapy procedure to the plurality of ablation therapy procedures; and add, to the database, data comprising the indication of the plurality of voltage pulses and the indication of the extrinsic characteristic; and a server coupled to the database, the server comprising: a server processor; and server memory comprising ML model training instructions, which when executed by the server processor cause the server processor to: query the database for data associated with a subset of the plurality of ablation therapies; receive query results from the database; generate a machine learning (ML) model training dataset from the query results; and train an ML model to generate an inference about an intrinsic characteristic of an ablation therapy procedure from extrinsic characteristics of the ablation therapy procedure.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1(A) and FIG. 1(B) illustrates an ablation therapy system 100 in accordance with examples of this disclosure. FIG. 1(C) illustrates one example of an irreversible electroporation waveform and one example of a high frequency irreversible electroporation waveform.

FIG. 2(A) and FIG. 2(B) illustrate probe pairs.

FIG. 3(A) illustrates a plot of voltage pulses and FIG. 3(B) illustrates a plot of measured current produced by the voltage pulses.

FIG. 4 illustrates an ablation therapy device 400 in accordance with examples of this disclosure.

FIG. 5 illustrates a routine 500 in accordance with examples of this disclosure.

FIG. 6 illustrates a routine 600 in accordance with examples of this disclosure.

FIG. 7(A) and FIG. 7(B) illustrate plot 702 and plot 708 that can be generated in accordance with examples of this disclosure.

FIG. 8 illustrates a plot 870 that can be generated in accordance with examples of this disclosure.

FIG. 9 illustrates a plot 850 that can be generated in accordance with examples of this disclosure.

FIG. 10 illustrates a routine 800 in accordance with examples of this disclosure.

FIG. 11 illustrates a plot 902 that can be generated in accordance with examples of this disclosure.

FIG. 12 illustrates a routine 1000 in accordance with examples of this disclosure.

FIG. 13 illustrates a routine 1100 in accordance with examples of this disclosure.

FIG. 14 illustrates a routine 1300 in accordance with examples of this disclosure.

FIG. 15(A) and FIG. 15(B) illustrate plot 1402 and plot 1408 that can be generated in accordance with examples of this disclosure.

FIG. 16 illustrates an ablation therapy system 1500 in accordance with examples of this disclosure.

FIG. 17 illustrates a technique 1600 in accordance with one examples of this disclosure.

FIG. 18(A) illustrates a graphical display in accordance with an example of this disclosure. FIG. 18(B) illustrates a graphical display in accordance with an example of this disclosure. FIG. 18(C) illustrates a graphical display in accordance with an example of this disclosure. FIG. 18(D) illustrates a graphical display in accordance with an example of this disclosure.

FIG. 19 illustrates a computer-readable storage medium 1800 in accordance with one embodiment.

FIG. 20 illustrates a diagrammatic representation of a machine 1900 in the form of a computer system within which a set of instructions may be executed for causing the machine to perform any one or more of the methodologies discussed herein, according to an example embodiment.

FIGS. 21(A)-21(B) illustrates plots that can be generated in accordance with examples of this disclosure.

FIG. 22 depicts a diagram illustrating the relationship between intrinsic/extrinsic factors and current.

FIG. 23 illustrates use of normalized current data to identify the transition between different zones of mechanisms of action.

FIG. 24 illustrates an exemplary machine learning (ML) system suitable for use an ablation therapy device in accordance with example(s) of the present disclosure.

FIG. 25(A) and FIG. 25(B) illustrates graphical displays that can be generated and displayed by an ablation therapy device in accordance with example(s) of the present disclosure.

FIG. 26(A) and FIG. 26(B) illustrate plots that can be generated by an ablation therapy device in accordance with example(s) of the present disclosure.

### DETAILED DESCRIPTION

The drawings, which are not necessarily to scale, depict selected embodiments and are not intended to limit the scope of the disclosure. The detailed description illustrates by way of example, not by way of limitation, selected embodiments.

The skilled artisan will readily appreciate that the devices and methods described herein are merely exemplary and that variations can be made without departing from the spirit and scope of this disclosure. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein the devices and methods for an ablation therapy system.

Proximal and distal refer to a direction or location relative to the patient's center. A proximal direction is course of movement away from the patient's center and toward the user. A proximal location is a position which further away from the patient's center and closer to the operator. A distal direction is a course movement toward the patient's center and away from the user. A proximal location refers to location further from the patient's center than a second location of the device during use. A distal location refers to a location nearer to the patient's center compared with a second location of the device during use.

Terms used herein should be accorded their ordinary meaning in the relevant arts, or the meaning indicated by their use in context, but if an express definition is provided, that meaning controls.

Herein, references to “one embodiment,” “an embodiment,” “one example,” “an example, or “embodiments” and “examples” in the plural do not necessarily refer to the same embodiment or require plural embodiments, although it may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

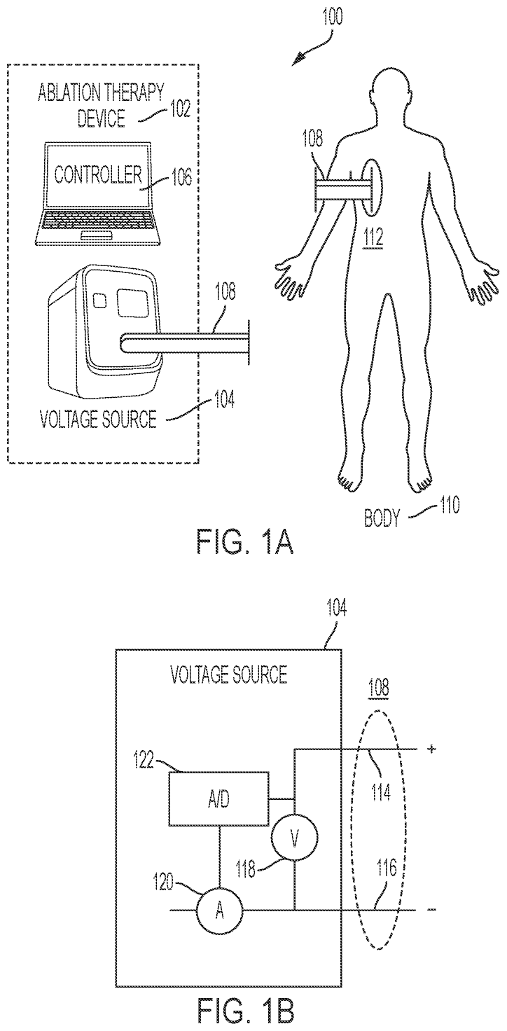

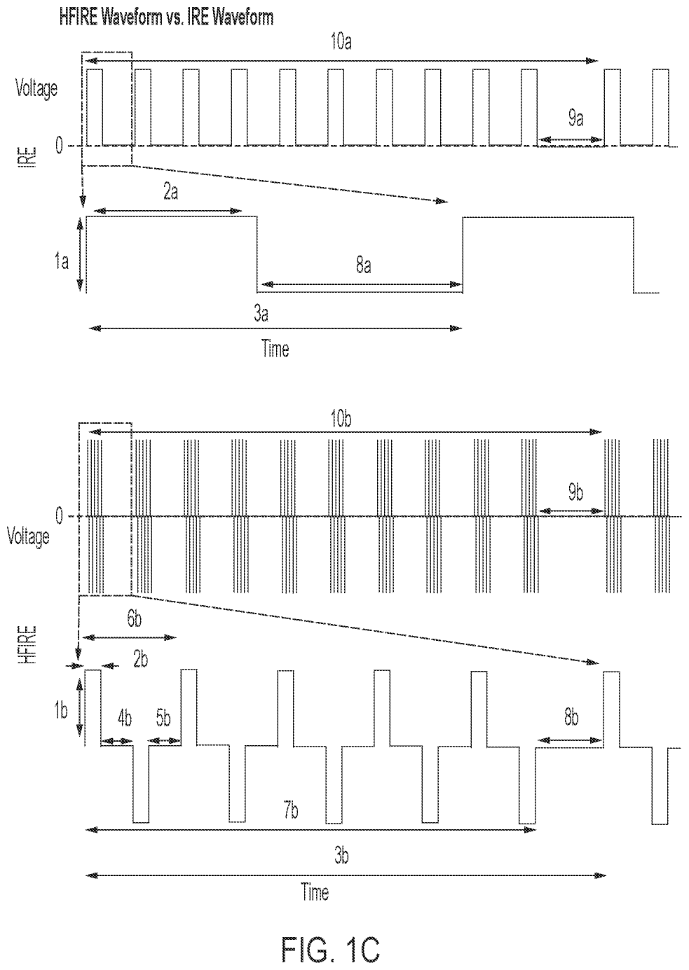

FIG. 1(A) illustrates an ablation therapy system 100. Ablation therapy system 100 includes an ablation therapy device 102 with which a clinician can apply an ablation therapy procedure to a body 110 of a patient. For example, a clinician can use the ablation therapy device 102 to apply an IRE or H-FIRE ablation therapy to body 110. Ablation therapy device 102 comprises a voltage source 104, a controller 106, and at least one probe 108. The voltage source 104 may comprise a generator configured to generate electrical pulses. The probe 108 may include, but is not limited to, a single monopolar probe with a single electrode (for example, up to 6 monopolar electrode probes may be used for a single IRE and/or H-FIRE treatment); a single bipolar probe comprising at least two electrodes; a single probe with multiple tines and each tine having either a single monopolar electrode or at least one tine having at least two electrodes; and/or a surface electrode with a single electrode. Table 3 below provides non-limiting examples of the various probe 108 and electrode configurations that may be used with the system.

TABLE 1

| | |
|---|---|
| Electrode type: | Parallel plate: |
| | 0.1 mm-70 cm diameter |
| | (and larger for applications relating to |
| | e.g., whole organ decellularization) |
| | Needle electrode(s): |
| | 0.001 mm-1 cm diameter |
| | Single probe with embedded disk |
| | electrodes: 0.001 mm-1 cm diameter |
| | Spherical electrodes: |
| | 0.0001 mm-1 cm diameter |
| | Needle diameter: 0.001 mm-1 cm |
| | Electrode length (needle): 0.1 mm to 30 cm |
| | Electrode separation: 0.1 mm to 5 cm, or |
| | even 5 cm to 20 cm, or 20 cm to 100 cm, |
| | and larger (for reversible electroporation, |
| | gene delivery, or positive electrode with |
| | ground patch on patient's exterior, e.g.) |
| Probe type: | Single monopolar probe with a single |
| | needle electrode; multiple monopolar |
| | probes (up to at least 6) with a single |
| | needle electrode; single bipolar probe with |
| | a bipolar needle electrode; multiple bipolar |
| | probes (up to at least 6) with a bipolar |
| | needle electrode; single catheter probe with |
| | multiple electrodes; single probe with |
| | multiple monopolar electrode tines; up to |
| | at least 6 surface electrode(s) with either a |
| | single surface electrode, multiple surface |
| | electrodes, or bipolar surface electrode(s); |
| | catheter comprising a circular loop and/or |
| | ring comprising a series of electrodes; |
| | catheter comprising an balloon with |
| | electrodes placed on a balloon outer |
| | surface; a catheter comprising an |
| | expandable flower shaped distal end with a |
| | series of electrodes |

In general, a clinician can use the ablation therapy device 102 to apply a series of voltage pulses to target tissue 112 of body 110 via the probe 108. The present disclosure can be applied to any type of target tissue 112, such as, for example, liver, prostate, kidney, pancreas, lung, head, neck, heart, brain, or other soft tissue areas of body 110. Although the present disclosure does not attempt to provide exhaustive examples of different ablation therapy protocols, a general ablation therapy is described. In general, an ablation therapy for which the present disclosure can be implemented will include application of a series of voltage pulses to target tissue 112. For example, voltage source 104 can repeatedly energize probe 108 to deliver a series of voltage pulses to target tissue 112.

In one aspect, at least a portion of the probe 108 can be configured for insertion into target tissue 112 of body 110 of a patient. Probe 108 can be any type including but not limited to the probes described in Table 3 above. Although not depicted, probe 108 can comprise a handle, a needle having a proximal end and a distal end, and at least one connector (e.g., to couple to voltage source 104). In some examples, the needle can comprise at least one electrode and a tip positioned at the distal end of the needle. The tip can be a sharp tip capable of piercing tissue of body 110. As used herein, probe 108 can include any number of pairs of probes. Alternatively, ablation therapy device 102 could include multiple probe 108 with which to form probe pairs. The term “probe pair” includes electrodes coupled to voltage source 104 and arranged to deliver a voltage to target tissue 112. It is noted that probe and electrode are often used interchangeably in this disclosure. Further, probe 108 can include multiple probe pairs. For example, probe 108 could include four probes which combined can be used to form 6 different pairs of probes.

Irreversible Electroporation (“IRE”) is a tissue ablation technique where high voltage electrical pulses are applied to target tissue in a target area or a treatment site. Included in IRE is High-Frequency Irreversible Electroporation (“H-FIRE”), which includes short electrical pulses commonly having a biphasic waveform, as described in more detail below. Consequently, current circulation through the target tissue can create an electrical field based on the spatial distribution of electrical properties of tissue which ultimately triggers different cell death mechanisms. In general, an IRE and/or H-FIRE protocol involves delivering a series of short and intense electric pulses through electrodes inserted directly into, or around, target tissue and/or on the surface of a patient's body. The pulses are designed to generate an electric field, between the electrodes, capable of inducing a rapid buildup of charge across the plasma membrane of cells. The charge across the plasma membrane of a cell is commonly referred to as a transmembrane potential (TMP).

Once the TMP reaches a critical voltage, it is thought that electrically conductive pores form in the membrane to prevent permanent damage by shunting current and limiting further TMP rise. If the pulse amplitude and duration are tuned to permit pore resealing, and cell viability is maintained following exposure, the process is categorized as reversible electroporation. However, where pore resealing does not take place, cell death occurs, and the process is categorized as IRE.

As known in the art, a target tissue has been successfully irreversibly electroporated when tissue cells are unable to seal the pores formed in the plasma membrane of the cells. A threshold voltage gradient (v/cm) and threshold number of pulses are required to achieve irreversible electroporation. It is within the conception of this disclosure to provide a user with a system and method to determine when these threshold parameters have been achieved and in turn an IRE and/or H-FIRE treatment transition point, or a certain therapy endpoint, has been achieved. The normalized current measurement provides clinicians with an indication of what ablation modality the target tissue is undergoing at a particular point in the treatment as well as transitions between ablation modalities. Electrical conductivity of the target tissue will rise upon the delivery of treatment pulses. The measurement of electrical conductivity is a known indicator for determining the extent of electroporation in tissue and can be used to determine if target tissue cells have been successfully irreversibly electroporated. However, direct measurement of the electrical conductivity of a target tissue is not a practical clinical approach as it highly depends not only on the shape factor (extrinsic property) but also on many other intrinsic properties which might vary during an IRE/HFIRE procedure. This makes direct measurements of electrical conductivity if not impractical very difficult to be used. The electrical conductivity measurements for HFIRE could be more complicated as it is frequency dependent. However, as will be described below in more detail, the normalized current may be analyzed during an IRE and/or H-FIRE treatment to determine an IRE and/or H-FIRE treatment endpoint.

H-FIRE protocols are comprised of bipolar and/or biphasic electrical pulses delivered at a higher repetition rate, as opposed to traditional IRE that uses unipolar and/or monopolar electrical pulses delivered at a lower repetition rate as compared to H-FIRE. Biphasic bursts of an H-FIRE protocol with inter-pulse delays can be used to ablate tissue while minimizing the need for paralytic needed to avoid muscle contractions often seen in traditional IRE treatments. Furthermore, high-frequency fields have the potential to overcome impedance barriers posed by low conductivity tissues, which could result in more homogenous and predictable treatment outcomes in heterogeneous systems.

High-frequency irreversible electroporation has also potential advantages for use in neurosurgery, including the ability to deliver pulses without inducing muscle contraction, inherent selectivity against malignant cells, and the capability of simultaneously opening the blood-brain barrier surrounding regions of ablation. The system comprises a voltage source 104 (see FIG. 1) capable of generating electrical pulse parameters capable of being used in either an IRE and/or H-FIRE treatment. Pulse parameters and other treatment parameters can be set as standardized parameters for typical IRE and/or H-FIRE treatments, specifically chosen by an end user prior to treatment, or may be changed by a treatment control module of the system during the treatment.

FIG. 1C shows representative IRE and HFIRE waveform graphs. Table 2 below provides a list of electrical pulse parameters that can be generated by the voltage source 104 and manipulated during ablation treatment procedures described herein:

TABLE 2

| Mechanism | RE | | IRE | | HFIRE | | Electrolysis | |
|---|---|---|---|---|---|---|---|---|
| Pulsing Polarity | Mono/Biphasic | | Monophasic | | Biphasic | | Mono/Biphasic | |
| Number of pulses | 1-50,000 | pulses | 1-50,000 | pulses | 1-50,000 | pulses | 1-50,000 | pulses |
| delivered: | | | | | | | | |
| Electric Field | <1000 | V/cm | 500-5,000 | V/cm | 500-5,000 | V/cm | 1,000-100,000 | V/cm |
| Density: | | | | | | | | |
| Frequency of Pulse | 0.001-1000 | Hz | 0.001-1000 | Hz | 0.001-1000 | Hz | 0.001-1000 | Hz |
| Application: | | | | | | | | |
| Frequency of pulse | 0-100 | MHz | 0-100 | MHz | 0-100 | MHz | 0-100 | MHz |
| signal: | | | | | | | | |

| Pulse shape/profile: | square, triangular, | | square, triangular, | | square, triangular, | | square, triangular, |
|---|---|---|---|---|---|---|---|
| | trapezoidal, | | trapezoidal, | | trapezoidal, | | trapezoidal, |
| | exponential | | exponential | | exponential | | exponential |
| | decay, sawtooth, | | decay, sawtooth, | | decay, sawtooth, | | decay, sawtooth, |
| | sinusoidal, alternating | | sinusoidal, alternating | | sinusoidal, alternating | | sinusoidal, alternating |
| | polarity | | polarity | | polarity | | polarity |
| Pulse type: | Positive, negative, | | Positive, negative, | | Positive, negative, | | Positive, negative, |
| | neutral electrode | | neutral electrode | | neutral electrode | | neutral electrode |
| | charge pulses (changing | | charge pulses (changing | | charge pulses (changing | | charge pulses (changing |
| | polarity within pulse) | | polarity within pulse) | | polarity within pulse) | | polarity within pulse) |
| | Multiple sets of pulse | | Multiple sets of pulse | | Multiple sets of pulse | | Multiple sets of pulse |
| | parameters for a | | parameters for a | | parameters for a | | parameters for a |
| | single treatment | | single treatment | | single treatment | | single treatment |
| | (changing any of the | | (changing any of the | | (changing any of the | | (changing any of the |
| | above parameters within | | above parameters within | | above parameters within | | above parameters within |
| | the same treatment to | | the same treatment to | | the same treatment to | | the same treatment to |
| | specialize outcome) | | specialize outcome) | | specialize outcome) | | specialize outcome) |
| Voltage (or Pulse | 1 V-3 kV | | 1 kV-10 kV | | 1 kV-10 kV | | 1 kV-100 kV |
| Amplitude) Applied: | | | | | | | |
| Pulse Width | 1-100 | μsec | 10-100 | μsec | 1-20 | μsec | 1 psec-1 min |

| | | | | | | | |
|---|---|---|---|---|---|---|---|
| Intraphase Delay | 1-10 | μsec | N/A | 1-10 | μsec | 1-10 | μsec |
| Interpulse Delay | 1-1000 | μsec | N/A | 1-1000 | μsec | 1-1000 | μsec |

| | | | | |
|---|---|---|---|---|
| Delay between | 1 psec-1,000 sec | 1 psec-1,000 sec | 1 μsec-1,000 sec | 1 μsec-1,000 sec |
| Trains | | | | |

A typical IRE waveform comprises a pulse amplitude 1a, a pulse width 2a, a pulse/burst interval 3a, a delay between pulses 8a, a delay between trains 9a, and a pulse/burst time 10a (i.e., the time of one train). Table 2 provides a list of electric pulse parameters that can be generated by the voltage source 104 and manipulated during treatment procedures discussed herein to achieve IRE ablation within a treatment site.

A typical HFIRE waveform comprises a pulse amplitude 1b, a pulse width 2b, a pulse/burst interval 3b, an intraphase delay 4b, an interpulse delay 5b, a bipolar pulse period 6b, a burst width 7b, a delay between bursts 8b, a delay between trains 9b, a and a pulse/burst time 10b (i.e., the time of one train). Table 2 provides a list of electric pulse parameters that can be generated by the voltage source 104 and manipulated during treatment procedures discussed herein to achieve HFIRE ablation within a treatment site:

The on time (i.e., the total time of energy delivery per burst) for HFIRE is equivalent to length of one IRE pulse (e.g., 100 μs). The bursts per minute for HFIRE is equivalent to pulses per minute for IRE (e.g., 90 BPM). The train is a set of bursts/pulses delivered consecutively before an extended delay.

Table 2 provides a list of electric pulse parameters that can be generated by the voltage source 104 and manipulated during a treatment procedure discussed herein to achieve Reversible Electroporation ablation within a treatment site.

Table 2 provides a list of electric pulse parameters that can be generated by the voltage source 104 and manipulated during a treatment procedure discussed herein to achieve Electrolysis ablation within a treatment site.

Some clinicians use a threshold change in current measurements during a treatment and/or after a treatment has completed to indicate a desired level of conductivity of the target tissue has been achieved, the impact of IRE or H-FIRE on the target tissue, and/or use a rise in current to indicate an end of treatment. However, intrinsic properties and extrinsic properties can affect the endpoint of treatment directly or indirectly. For example, tissue specific properties (intrinsic property) can affect the current response and therefore the end of treatment. As another example, the probe exposure (extrinsic property) has great impact on the tissue ablation volume and the current trend. For clarity, the current measurements used by clinicians currently known in the art are not the normalized current measurement as described herein. Current can be directly measured by commercial devices and current is a direct output measurement of the tissue being treated. Moreover, measuring current intrinsically includes all of the unique tissue characteristics as well as unique system input parameters (voltage, pulse trains, etc.) for each treatment. It is also known that electrical conductivity of tissue in a treatment site is a useful parameter to be studied before and after IRE and/or H-FIRE treatment in order to determine a treatment endpoint.

It is known in the art to look for a target amp rise in the measured current during an IRE and/or H-FIRE procedure to indicate complete irreversible electroporation of the tissue within the target site and an end of treatment. However, it should be noted that in a multi-probe configuration, the last 2-3 probe pairs may not achieve this target amp rise in current because of treatment overlap, and effective electroporation has already occurred in tissue proximate to the last probes. Another factor complicating methods of analyzing IRE and H-FIRE treatment protocols is that output current is voltage dependent. Therefore, increasing voltage for a probe pair might result in the desired target amp rise in current but that is due to the ohmic effect and not due to electroporation. As such, the observed rise in current may not translate to actual ablation volume or treatment efficacy.

Using conductivity changes as an indication of the extent of electroporation during a treatment may be problematic due to the intrinsic and extrinsic factors unique to a particular procedure. Specifically, voltage and the shape factor impact dynamic conductivity measurements. Equation 1 detailed below illustrates the relationship between normalized current and normalized conductivity, where V=voltage, I=current, S=shape factor, σ=conductivity, and subscript ₀ denotes initial value.

$$\frac{I}{{I}_{0}} = \frac{S ⨯ σ ⨯ V}{{S}_{0} ⨯ {σ}_{0} ⨯ {V}_{0}} = \frac{σ}{{σ}_{0}} \qquad Equation 1$$

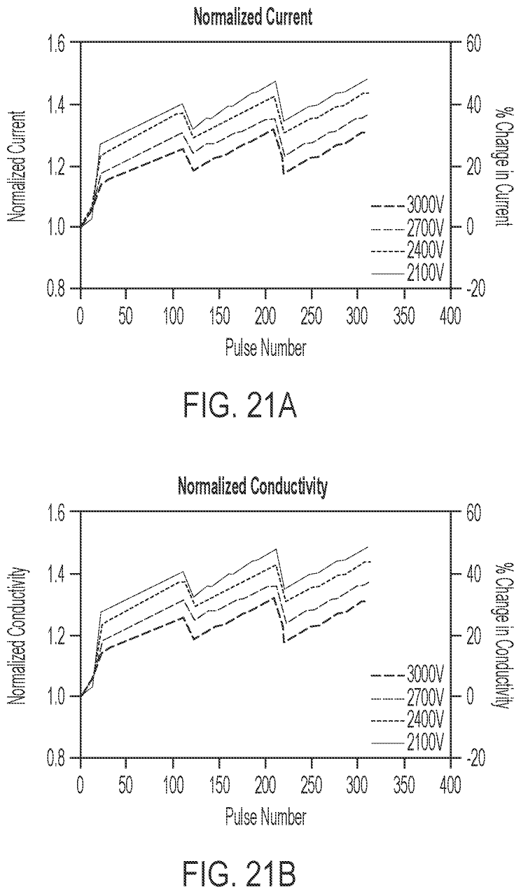

Thus, when the shape factor (S) and voltage (V) are held constant during a procedure, normalized current and normalized conductivity will be equal. FIGS. 21(A)-21(B) illustrates the relationship between conductivity and current changes for a single procedure. FIG. 21(A) is a plot depicting changes in the normalized current and FIG. 21(B) is a plot depicting changes in normalized conductivity. Voltage pulses are depicted on the X axis, normalized values on left Y axis and percent change on the right Y axis. Separate trend lines are shown for 2100, 2400, 2700 and 3000 voltages. The trend lines and individual data points of FIGS. 21 (A) and 21 (B) are identical, illustrating that only when voltage and the shape factor remain constant during a procedure is conductivity and current the same and an accurate indication of tissue changes due to electrical pulses. Because the shape factor value may vary during the IRE/HFIRE onset (e.g., changes made during a procedure such as probe position, deactivation of a probe pair, and/or modification to exposure length), analysis of the conductivity trends for procedure planning and/or monitoring is not as reliable as an analysis of current trends. This is due to the fact that to be able to estimate the conductivity a set of assumptions are needed, which might not be valid before, during and after the IRE/HFIRE procedure.

An advantage of using the normalized current for treatment planning purposes, as described in more detail herein, is that current inherits the characteristics of extrinsic factors and/or intrinsic factors for individual treatment procedures. For example, as described in more detail below, extrinsic factors and intrinsic factors comprise different patient specific and/or treatment specific parameters. Each individual ablation procedure will comprise a unique set of extrinsic factors and intrinsic factors. These unique extrinsic factors and intrinsic factors will be accounted for as inherent features of measuring the current and then normalizing the current (as described herein). Therefore, each unique extrinsic factor and/or intrinsic factor for an individual treatment procedure will be inherit in the normalization of the current for treatment planning purposes.

Accordingly, this disclosure satisfies a need in the art to create a system and reliable method to determine the efficacy of ablation from IRE or H-FIRE and consequently determine a completion to the treatment protocol.

Furthermore, given the number of parameters of an IRE or H-FIRE treatment protocol (e.g., voltage level, number of pulses, pulse polarity, pulse length, delay between pulses, or any of the various pulse parameters in Table 2) it is difficult to compare current output from one treatment to another. These hurdles make comparing results of different treatment protocols difficult as the individual characteristics of the patient coupled with the numerous parameters in an IRE or H-FIRE treatment protocol preclude simply comparing results based on the current measured during the treatment. This difficulty extends to comparing IRE or H-FIRE treatments across clinicians or across treatment centers. These difficulties create problems and inefficiencies in running pilot studies or sharing treatment results between clinicians or clinics. Therefore, a need in the art exists to simplify and accurately compare current output data across various treatments, different treatment protocols, and across different clinicians and/or treatment centers.

For example, the current between two probes may vary based on a number of factors (e.g., voltage, number of pulses, pulse length, delay between pulses, or any of the various pulse parameters in Tables 1). Complicating this, current depends on various intrinsic properties of the individual patients (blood perfusion and the extent of vascular structures, thermal properties of tissue, electrical properties of tissue) and extrinsic properties of each treatment (pulsing parameters (IRE or HFIRE), shape factor, probe placement parameters).

As will be discussed in more detail below, collecting, analyzing and/or comparing normalized current data during an IRE and/or H-FIRE treatment can be used to provide an end user sufficient information to determine when the cumulative electrical pulses sufficiently result in irreversible electroporation of the tissue within the treatment site. Therefore, normalizing current data may be used to determine a treatment endpoint, the endpoint of a treatment zone, and/or to plan an effective IRE and/or H-FIRE treatment.

Intrinsic factors are associated with target tissue properties including tissue type, cell size, cell homogeneity, tissue perfusion levels, tissue conductivity and temperature. Extrinsic factors are not related to tissue characteristics but do impact tissue response to the ablation therapy. Extrinsic factors are related to the delivery of electrical fields to the tissue include probe configuration, ablation volume, applied voltage, and specific pulse parameters, among others. The relationship between intrinsic/extrinsic factors and current is conceptually depicted in FIG. 21. Extrinsic factors 1 result in the generation of electrical energy which, when applied to the target tissue 2, result in a measurable current output 3. In other words, measured current inherits the all the unique and complex intrinsic and extrinsic characteristics which define a tissue response to electrical energy. Although electrical conductivity is an important property and affects results of an electroporation treatment, it is highly dependent on intrinsic properties which might vary during the procedure. However, current inherits all these complexities and reflects what electrical energy does to tissue without assumptions, error propagation or oversimplification which might be needed for to determine electrical conductivity.

The relationship between current, the intrinsic factors, and the extrinsic factors can be expressed mathematically. Irreversible electroporation (IRE) using a monophasic waveform, high-frequency irreversible electroporation (HFIRE) using a biphasic waveform, and or reversible electroporation (RE) using a standard RE waveform is represented by Equation 2. Equation 3 describes current dependence on shape factor and electrical conductivity, which are products of the electrode placement/shape and nature of the target tissue accordingly. Where L=length of probe exposure; r=radius of the probe; V=voltage across the probes; d=distance between probes; t=total timing period; S=shape factor; a (x, y, z, t)=electrical conductivity of tissue; k (x, y, z, t)=thermal conductivity of tissue; ω=blood perfusion; Cₚ=specific heat of tissue; X=anisotropic factor; waveform=polarity, pulse width.

I[A]=f(L,V,d,S,t,σ(x,y,z,y),k(x,y,z,t),Cₚ,ω,X,IRE/HFIRE/RE waveform . . . ) Equation 2

I[A]=S(L,d,r,X, etc.)×σ(ω,K,Cₚ,X, etc.)×V Equation 3

A benefit to the present disclosure, or said differently, to representing current using equations given herein and normalizing the current to compare current across treatments is that the present disclosure can be used to simplify the impact of these numerous variables associated with ablation volume from IRE and H-FIRE treatments. In one embodiment, the lack of interaction between the current trend for different voltages suggests that the success of IRE is independent of the tested voltages used in treatment planning. In one embodiment, the normalization of current begins with the first pulse of an ablation procedure and upon the initial current which is measured during the first pulse. The normalized current calculation occurs continuously throughout the treatment as the treatment progresses. The normalized current calculation should be treated for individual probe pairs, as each probe pair has its individual characteristics (i.e., exposure, spacing, voltage, etc.).

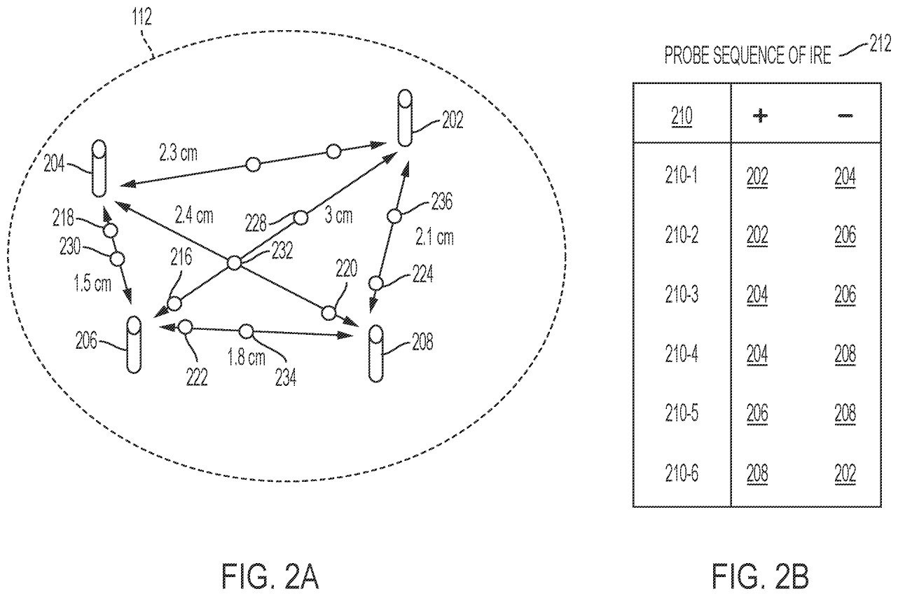

As discussed herein, the normalization of current inherently accounts for changes to specific extrinsic factors or intrinsic factors for a single procedure, for different procedures of the same patient, or for different procedures for different patients. In one embodiment, as shown in FIG. 2(A) and FIG. 2(B) a series of electrical pulses are delivered between a series of four probes. For example, probe 202, probe 204, probe 206, and probe 208 which can be grouped into pair of probes 210. The probes 210 can be inserted into target tissue 112. The probes can be placed a variety of distances apart. The distances depicted in FIG. 2(A) are shown for example only. In general, the distance between probes may depend upon the type of target tissue 112, the size or area of target tissue 112, the total number of probes used in the therapy. A clinician can place probe 108 into body 110 such that probes (or electrodes) of probe 108 are inserted into desired locations within or proximate to target tissue 112. The normalization of current can be derived from this specific pulse paradigm (and transferred to a treatment database as described in more detail below) with extrinsic factors or intrinsic factors, such as the number of probes, specific probe placement, electrode exposure, the number of pulses delivered between each probe pair, or the specific cycling of probe activation being inherent within the normalized current.

In another embodiment, different pulse paradigms may be applied and extrinsic factors and/or intrinsic factors for each of these various pulse paradigms will be inherent in the normalized current. For example, regardless of the specific pulse paradigm used by a physician, the normalization of current will inherently account for the differences between the extrinsic factors and intrinsic factors across the following pulse paradigms: (i) a non-cycled pulse paradigm 100 pulses were delivered per electrode pair for a total number of 600 pulses to the target tissue; (ii) a cycled pulse paradigm (5 pulse cycle, 0s delay scheme), where 20 pulses were delivered per electrode pair, yield 120 total pulses per cycle and, again, a total of 600 pulses to the target region; (iii) a cycled pulse paradigm (5 pulse cycle, 0s delay scheme), where 20 pulses were delivered per electrode pair, yield 120 total pulses per cycle and, again, a total of 600 pulses to the target region with an enhanced electrode pair activation pattern such that no single electrode was activated more than two consecutive times; (iv) a non-cycled pulse paradigm 100 bursts of pulses were delivered per electrode pair for a total number of 600 bursts of pulses to the target tissue; (v) a cycled pulse paradigm (5 pulse cycle, 0s delay scheme), where 20 burst of pulses were delivered per electrode pair, yield 120 total burst of pulses per cycle and, again, a total of 600 bursts of pulses to the target region; (vi) an asymmetric bipolar waveform and/or monopolar waveform where the positive pulses and/or negative pulses have different durations (each pulse duration ranging between 0.25 μs to 2 μs); or (vii) an asymmetric bipolar waveform where the intrapulse delay varies or where there is no intrapulse delay.

An ablation therapy protocol (or treatment) can include applying a series of voltage pulses via each of the pairs from the pair of probes 210. In some examples, between 10 and 100 voltage pulses can be delivered via each probe pair. With some ablation therapy protocols, voltage pulses are applied via the pair of probes 210 in a sequential order. More particularly, all voltage pulses are applied via the first pair of probes, followed by the second pair of probes, etc. Further, applying voltage pulses can be repeated over a number of rounds. For example, a hypothetical ablation therapy protocol could include applying a specific number of voltage pulses having a specific magnitude via the first pair of probes 210-1, applying the specific number of voltage pulses having the specific magnitude via the second pair of probes 210-2, and so forth until the last pair of probes 210. This could be referred to as a first round of treatment. A therapy could include multiple rounds. The voltage and the number of pulses need not be the same between rounds. A user may physically move, realign, and/or reposition the placement of the probes within a patient (or for surface electrodes on a patient) between rounds.

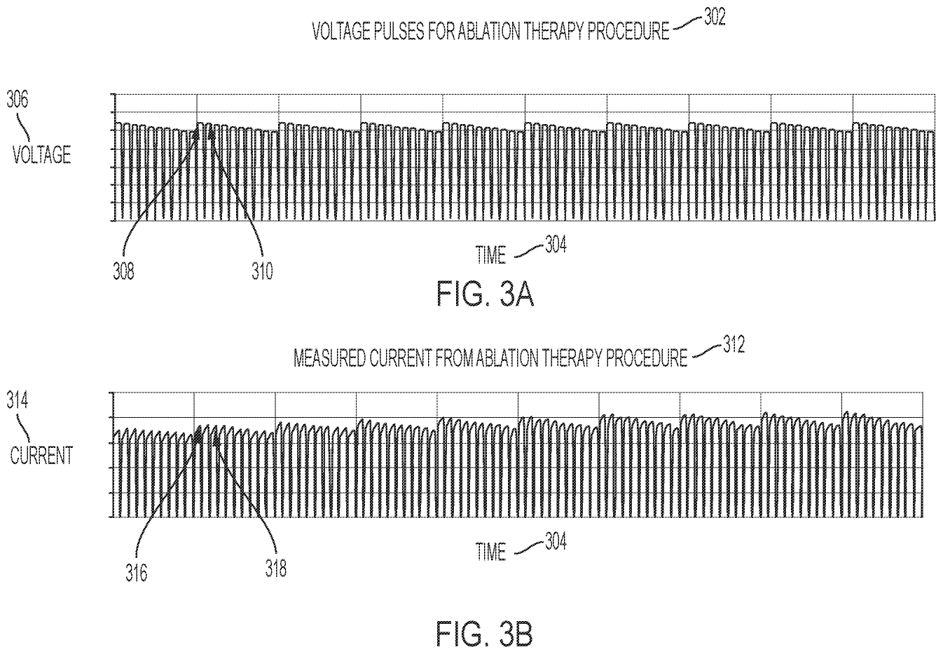

Referring back to FIG. 1, voltage source 104 can be any of a variety of energy sources capable of generating a voltage potential between respective positive and negative electrodes (not shown). Voltage source 104 can apply the generated voltage potential as a series of pulses to target tissue 112 via probe 108. For example, FIG. 3(A) depicts a plot showing a series of voltage pulses 302 where time 304 is represented on the X axis and voltage 306 on the Y axis. It is noted that time 304 and voltage 306 are not depicted to scale in this figure. The series of voltage pulses 302 includes a number of voltage pulses. For example, individual voltage pulse 308 and individual voltage pulse 310 are called out. FIG. 3(B) illustrates a plot showing a series of measured current 312 where time 304 is represented on the X axis and current 314 on the Y axis. Like the plot in FIG. 3(A), time 304 and current 314 are not depicted to scale in FIG. 3(B). As depicted, the series of measured current 312 of delivered electrical pulses includes a number of pulses which each correspond to one of the voltage pulses from the series of voltage pulses 302. For example, individual current measurement 316 and individual current measurement 318 are called out. As described more fully herein, ablation therapy device 102 can measure current produced by applying the series of voltage pulses 302 to target tissue 112, for example, resulting in the series of measured current 312. Examples directed to normalizing this measured current and techniques and devices configured to respond to the normalized current are more fully described below.

Voltage source 104 can, in some examples, be arranged to generate a voltage potential of up to 10,000 Volts. It is within the conception of this disclosure that the voltage source 104 is capable of achieving the various ranges of pulse parameters and/or probe embodiments described in Table 2 above. As way of a non-limiting example, voltage source 104 can be arranged to deliver the voltage potential as a series of pulses where each pulse can have a pulse width of up to 100 μsec. Furthermore, a delay or dwell between voltage pulse up to 2,000 msec (actual delay may depend on cardiac synchronization and/or patient pulse rate) and bursts on time can be up to 200 microseconds. Voltage source 104 can be powered by an A/C power source, D/C power source including a battery. The battery can be rechargeable. For example, voltage source 104 can include an A/C power source arranged to power voltage source 104 where access to A/C power (e.g., 110V, 240V, or the like) is available and to charge the battery such that the battery can power the voltage source 104 where access to A/C power is not available.

Controller 106 can be any of a variety of computing devices coupled to voltage source 104. A clinician can configure the ablation therapy device 102 for a particular ablation therapy protocol. For example, controller 106 can receive input from a clinician associated with the number of probes, the probe pair sequence, the desired voltage, the desired number of pulses, or the like and can send control signals to the voltage source 104 to cause the voltage source to apply voltage pulses the target tissue 112 via probe 108. By way of a non-limiting example, U.S. Publication US2016/0354142, filed Aug. 17, 2016, describes a controller system to be used in combination with the systems, devices, and methods described herein and is incorporated herein by reference.

Additionally, controller 106 can receive indications of current produced by application of the voltage pulses to the target tissue 112 by probe 108. Controller 106 can normalize the current and send control signals to the voltage source based on the normalized current. This and other examples are described more fully herein.

FIG. 1(B) illustrates a portion of system 100 of FIG. 1(A). As noted, system 100 is provided to measure current during an IRE or H-FIRE procedure and to normalize the current (among other examples). As illustrated in FIG. 1(B), system 100 includes a probe 108 having a positive electrode 114 and a negative electrode 116. Furthermore, system 100, and particularly, voltage source 104 includes a voltage sensor 118 and a current sensor 120. In general, the voltage sensor 118 can be connected across the positive electrode 114 and the negative electrode 116 (e.g., in parallel with the target tissue 112, or the like) while the current sensor 120 can be connected in series with one of the electrodes (e.g., the negative electrode 116 in this case).

Voltage source 104 can further include an analog to digital (A/D) converter 122 coupled to the voltage sensor 118 and the current sensor 120. A/D converter 122 can further be coupled to controller 106. Sensed values may be periodically, repeatedly, or continuously received and digitized by A/D converter 122 and transmitted to controller 106. In some examples, A/D converter 122 can sample the sensed values at rate of greater than 100 MHz.

With some examples, voltage sensor 118 can be a voltage divider, such as, comprised of two serially connected resistors, which measures a voltage drop across a known resistance value. The voltage sensor 118 can use resistors that are of much higher resistance than the tissue. As a specific example, the resistors in voltage sensor 118 can be in the kiloohm (ku) to megaohm (Me) range whereas target tissue 112 may typically have a resistance in the hundreds of ohms (a). As such, the voltage sensor may have a negligible voltage drop relative to the target tissue.

In some examples, the current sensor 120 can be a Hall effect sensor positioned around an electrode so as to measure electric current without directly interfering with the voltage pulse. Typically, the current sensor 120 is placed on the negative side (e.g., negative electrode 116) of the pair of electrodes in probe 108. Where more than two electrodes are present, multiple current sensors 120 can be provided.

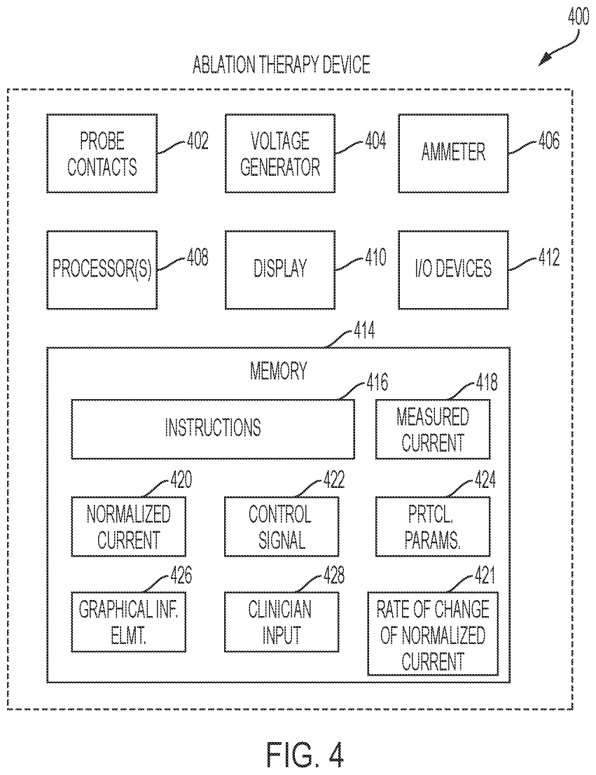

FIG. 4 illustrates an ablation therapy device 400. In some examples, ablation therapy device 400 can be implemented as the ablation therapy device 102 of FIG. 1. Ablation therapy device 400 includes probe contacts 402, voltage generator 404, ammeter 406, processor(s) 408, display 410, input and/or output devices (I/O devices 412), and memory 414. Probe contacts 402 can include mechanical coupling mechanisms to secure probes (e.g., probe 108, or the like) to the ablation therapy device 400. Further probe contacts 402 includes electrical contacts to provide electrical conductivity between voltage generator 404 and electrodes in a probe. Voltage generator 404 can be any of a variety of voltage generators arranged to generate voltage pulses as described herein. A discussion of voltage generators and voltage pulses was given above with respect to FIG. 1 and is not repeated here for brevity. Ammeter 406 can be any of a variety of current measuring devices electrically coupled to voltage generator 404 and/or probe contacts 402 such that current produced by application of voltage pulses to target tissue can be measured.

The processor(s) 408 can include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor(s) 408 may include graphics processing portions and may include dedicated memory, multiple-threaded processing and/or some other parallel processing capability. In some examples, the processor(s) 408 may be an application specific integrated circuit (ASIC) or a field programmable integrated circuit (FPGA). In some implementations, the processor(s) 408 may be circuitry arranged to perform particular computations, such as, related to artificial intelligence (AI) or graphics. Such circuitry may be referred to as an accelerator. Processor(s) 408 can include multiple processors, such as, for example, a central processing unit (CPU) and a graphics processing unit (GPU).

The memory 414 can include both volatile and nonvolatile memory, which are both examples of tangible media configured to store computer readable data and instructions to implement various embodiments of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), dynamic random access memory (DRAM), NAND memory, NOR memory, phase-change memory, battery-backed volatile memories, networked storage devices, and the like.

The memory 414 may include a number of memories including a main random access memory (RAM) for storage of instructions and data during program execution and a read only memory (ROM) in which read-only non-transitory instructions are stored. Memory 414 may include a file storage subsystem providing persistent (non-volatile) storage for program and data files. Memory 414 may further include removable storage systems, such as removable flash memory.

The memory 414 may be configured to store the basic programming and data constructs that provide the functionality of the disclosed processes and other embodiments thereof that fall within the scope of the present disclosure. Memory can store instructions 416, measured current 418, normalized current 420, rate of change of the normalized current 421, control signal 422, protocol parameters 424, graphical information element 426, and clinician input 428. During operation, processor(s) 408 can read instructions 416 from memory 414 and can execute the instructions 416 to implement embodiments of the present disclosure. Memory 414 may also provide a repository for storing data used by the instructions 416 or data generated by execution of the instructions 416.

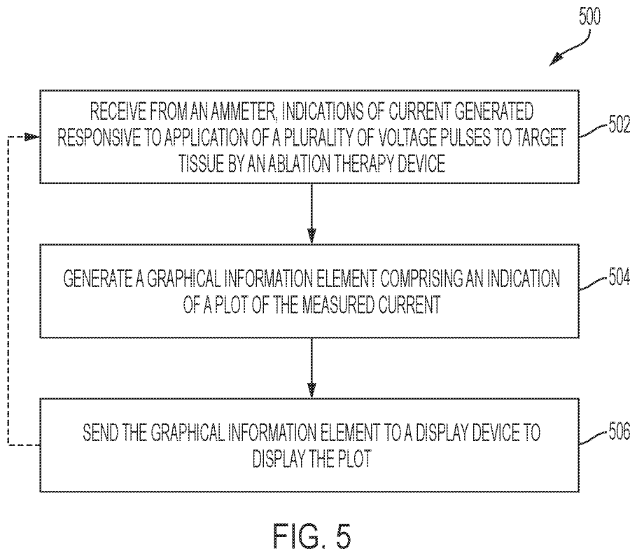

FIG. 5 depicts a routine 500 that may be implemented by an ablation therapy device according to examples of the present disclosure. At block 502 “receive from the ammeter, indications of current generated responsive to application of a plurality of voltage pulses to the target tissue by an ablation therapy device” indications of current measured at an ammeter can be received. For example, in executing instructions 416 processor(s) 408 can receive measured current 418 from ammeter 406.

At block 504 “generate a graphical information element comprising an indication of a plot of the measured current” graphical data comprising an indication of a plot of the measured current 418 can be generated. For example, in executing instructions 416 processor(s) 408 can generate graphical data (e.g., display frames, or the like) including indications of a plot representing the current measured at block 502 (e.g., measured current 418). The graphic data can be stored in memory 414 as graphical information element 426.

At block 506 “send the graphical information element to a display device to display the plot” the ablation therapy device can send the graphical information element 426 to display 410 to display the plot indicated by the graphical information element 426. For example, in executing instructions 416 processor(s) 408 can send the graphical information element 426 to display 410 and display 410 can display the plot indicated by the graphical information element 426.

In some examples, routine 500 can be repeated such that display 410 can be updated with indications of measured current 418 as an ablation therapy treatment progress. For example, ablation therapy device 102 could implement routine 500 at the conclusion of each round of voltage pulses, at the conclusion of each subset or train (e.g., 5 pulses, 10 pulses, 20 pulses, or the like) of voltage pulses. As such, display 410 can be updated with indications (e.g., via plots, or the like) of measured current 418 as the ablation therapy progresses providing feedback to the clinician of the progress of the ablation therapy treatment prior to a conclusion of the specified treatment protocol (e.g., prior to application of all scheduled voltage pulses, or the like).

Further, routine 500 can be repeated individually for each pair of probes or collectively for all probe pairs. For example, routine 500 can be implemented such that a plot depicting current from one pair of probes can be generated at block 504 and can be repeated such that another plot depicting current from another pair of probes can be generated at block 504. In some examples, both plots can be displayed on display 410. In other examples, a single plot depicting current from multiple pairs of probes can be generated.

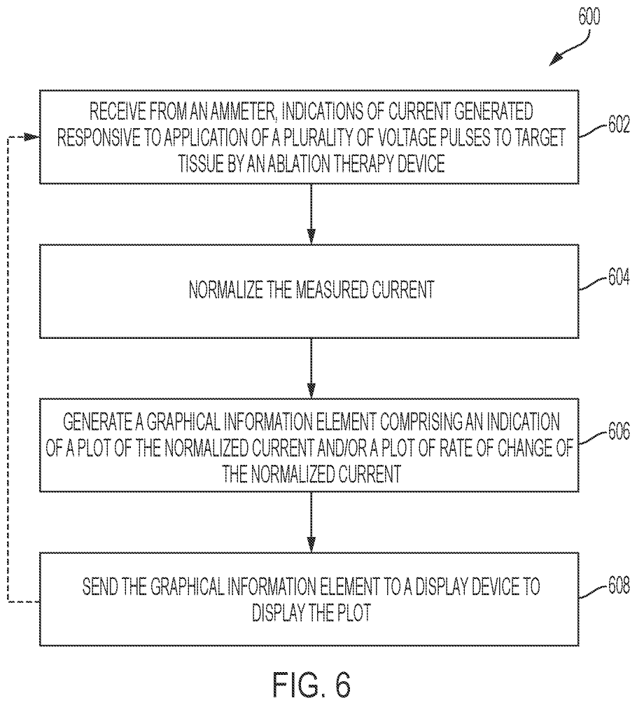

FIG. 6 depicts a routine 600 that may be implemented by an ablation therapy device according to examples of the present disclosure. At block 602 “receive from the ammeter, indications of current generated responsive to application of a plurality of voltage pulses to the target tissue by an ablation therapy device” indications of current measured at an ammeter can be received. For example, in executing instructions 416 processor(s) 408 can receive measured current 418 from ammeter 406.

At block 604 “normalize the measured current” the measured current can be normalized. For example, in executing instructions 416 processor(s) 408 can normalize the measured current 418 to generate normalized current 420. In some examples, processor(s) 408 can execute instructions to normalize the measured current for voltage. Said differently, processor(s) 408 can execute instructions 416 to normalize measured current 418 to a common reference point, resulting in normalized current 420. With some examples, current can be normalized with any normalization techniques such as linear scaling, clipping, log scaling or Z-score, or other statistical normalization techniques. As a specific example, measured current can be normalized using Equation 4, where I′=normalized current; I₀=initial current; I=final current after 10 pulses.

I′=I[A]/I₀[A] Equation 4

In some examples, at block 604 a rate of change of the normalized current can be derived. For example, processor(s) 408 in executing instructions 416 can determine a rate of change of the normalized current 420 using Equation 5, where RC=rate of change of current; I′=normalized current; t=time.

RC=dI′/dt Equation 5

At block 606 “generate a graphical information element comprising an indication of a plot of the normalized current” graphical data comprising an indication of a plot of the normalized current can be generated. For example, in executing instructions 416 processor(s) 408 can generate graphical data (e.g., display frames, or the like) including indications of a plot representing the normalized current 420. The graphic data can be stored in memory 414 as graphical information element 426. With some examples, the graphical information element 426 can include indications of a plot depicting the normalized current 420, a derived rate of change of the normalized current 420, or both the normalized current 420 and a derived rate of change of the normalized current 420.

At block 608 “send the graphical information element to a display device to display the plot” the ablation therapy device can send the graphical information element 426 to display 410 to display the plot indicated by the graphical information element 426. For example, in executing instructions 416 processor(s) 408 can send the graphical information element 426 to display 410 and display 410 can display the plot indicated by the graphical information element 426.

In some examples, routine 600 can be repeated such that display 410 can be updated with indications of normalized current 420 as an ablation therapy treatment progresses. For example, ablation therapy device 102 could implement routine 600 at the conclusion of each round of voltage pulses, at the conclusion of each subset or train (e.g., 5 pulses, 10 pulses, 20 pulses, or the like) of voltage pulses. As such, display 410 can be updated with indications (e.g., via plots, or the like) of normalized current 420 as the ablation therapy progresses providing feedback to the clinician of the progress of the ablation therapy treatment prior to a conclusion of the specified treatment protocol (e.g., prior to application of all scheduled voltage pulses, or the like).

Further, routine 600 can be repeated individually for each pair of probes or collectively for all probe pairs. For example, routine 600 can be implemented such that a plot depicting normalized current from one pair of probes can be generated at block 606 and can be repeated such that another plot depicting normalized current from another pair of probes can be generated at block 606. In some examples, both plots can be displayed on display 410. In other examples, a single plot depicting normalized current from multiple pairs of probes can be generated.

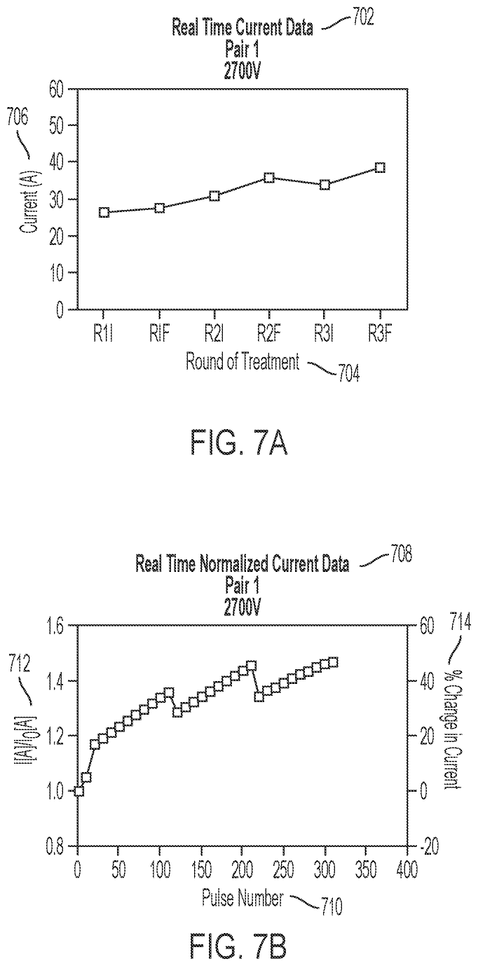

FIG. 7(A) is a plot 702 depicting real time current data for a probe pair over a number of rounds 704 of an ablation therapy treatment. A treatment round is a group of pulses delivered between a pair of probes before moving to the next pair. For example, in one embodiment round 1 range between 10 pulses to 20 pulses, and subsequent rounds are between 70 pulses to 100 pulses. However, the number of pulses in each round could be different depending on the physician's specific treatment protocol. The rounds of treatment 704 are depicted on the X axis while the current 706 is depicted on the Y axis. For example, R1I represents round 1 initial pulse and R1F represents the round 1 final pulse before moving to other probe pairs, and R2I represents round 2 initial pulse and R2F represents the round 2 final pulse before moving to other probe pairs. With some examples, routine 500 can generate graphical information element 426 including indication of a plot like plot 702. Both intrinsic and extrinsic factors may cause variation in tissue response from one probe pair to another.

FIG. 7(B) is a plot 708 depicting real time normalized current data for a probe pair over a number of voltage pulses of an ablation therapy treatment. The voltage pulse 710 are depicted on the X axis while the normalized current 712 and the percent change 714 are depicted on the Y axis. With some examples, routine 600 can generate graphical information element 426 including indication of a plot like plot 708. The data depicted in FIG. 7(A), which reflects the unique current/conductivity profile for a specific probe pair in a specific tissue type, can be used by the clinician to adjust pulse delivery to that probe pair to account for these variations. FIG. 7(B) illustrates the normalization of the current data depicted in FIG. 7(A), and as such provides the user with data that can be used for treatment planning purposes and real time monitoring of treatment progress, as described in more detail below.

An IRE and/or H-FIRE treatment procedure can include multiple therapy zones. In general, as used herein, a therapy zone is region associated with a particular treatment characteristic (e.g., a trend in tissue conductivity, a trend in measured current, a trend in normalized current, or the like). A therapy zone is representative of the specific mechanism(s) of action causing or resulting in changes at the cellular level due to the delivery of the electrical pulses. For example, a therapy zone may comprise any of the following, reversible electroporation (RE), irreversible electroporation (IRE), high frequency irreversible electroporation (HFIRE), thermal ablation, electrolysis, RE and IRE and HFIRE, IRE and HFIRE, IRE and HFIRE and thermal ablation and electrolysis, and/or thermal ablation and electrolysis. A particular therapy zone may be associated with a particular tissue response—for example irreversible electroporation of tissue within the target zone. Some clinicians desire to conclude an ablation therapy treatment in one of these therapy zones or at a transition between selected therapy zones. However, given conventional ablation therapy tools and treatment protocols there is not a way to determine which therapy zone the treatment is currently in or to predict how the treatment will progress through the therapy zones.

In one embodiment, zone one, zone two, and zone three can have different intensity levels of IRE and/or HFIRE. For example, zone one has a lower IRE/HFIRE intensity than zone two and zone three; whereas zone three has a high IRE/HFIRE intensity than zone two and zone one. The different IRE and/or HFIRE intensities affect the tumor microenvironment with different mechanisms of action.

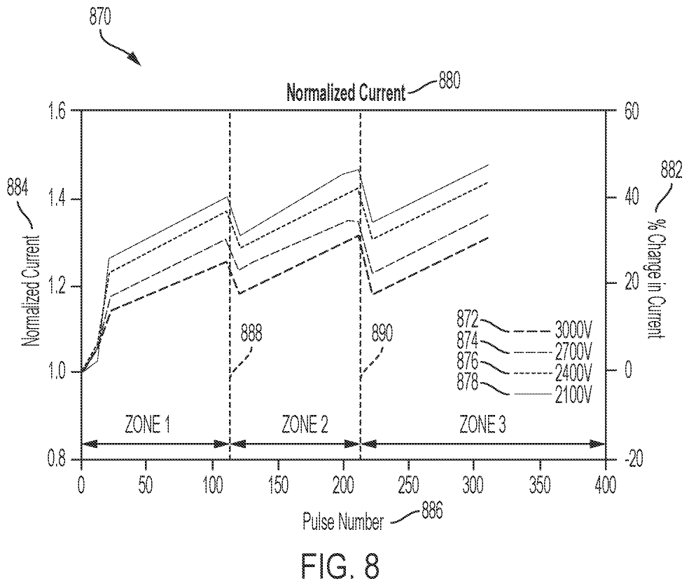

In one embodiment, the specific and/or predominate mechanism of action causing or resulting in changes at the cellular level in each specific zone(s), with reference to FIG. 8, is depicted in Table 3 below:

TABLE 3

| | Zone Zero | Zone One | Zone Two | Zone Three |
|---|---|---|---|---|
| Potential | RE, | RE, | RE, IRE, | Thermal |
| mechanism(s) | IRE, and/or | IRE and/or | HFIRE, | ablation |
| of action: | HFIRE | HFIRE | thermal | and/or |
| | | | ablation, | electrolysis |
| | | | and/or | |
| | | | electrolysis | |
| Predominate | RE | IRE and/or | IRE and/or | Thermal |
| mechanism(s) | | HFIRE | HFIRE | ablation |
| of action: | | | | and/or |
| | | | | electrolysis |

The specific and/or predominate mechanism of action of cell death in each zone will be dependent on certain extrinsic and/or intrinsic factors (i.e., tissue type, conductivity of the target area, pulse paradigm, specific pulsing patters, applied voltage).

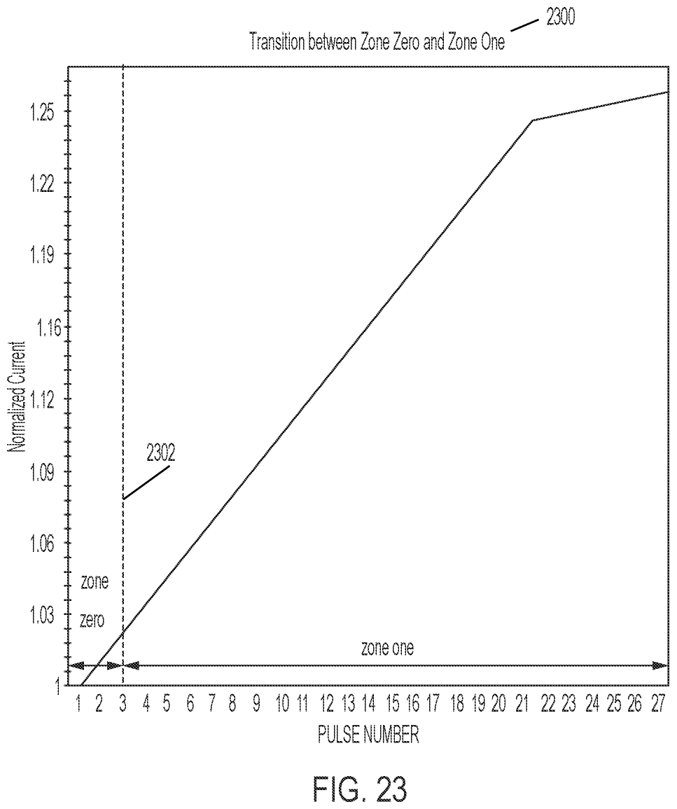

In one embodiment, in zone zero the predominate mechanism of action is RE with potentially IRE and/or HFIRE effects. The specific transition point between mechanism of action predominantly RE and predominantly IRE/HFIRE (for one example, see FIG. 23) may be impacted by the heterogeneity of electric field (e.g., using needle electrodes) and/or the heterogeneity of tissue (i.e., different cell types: epithelial, healthy, cancerous). For example, some cells might need more pulses to achieve the voltage gradient sufficient to achieve IRE and/or HFIRE (i.e., greater than 800 v/cm) and for cells in target area to form irreversible pore formation. In zone one, the mechanism of action transitions to predominantly IRE and/or HFIRE. In zone one, the vasculature structure may be preserved for continued blood supply and oxygenation to the target site. The extent of apoptotic response as a result of the IRE and/or HFIRE would likely induce anti-inflammatory cell death in cancerous cells while preserving the extracellular membrane structure. In zone two, the mechanism of action transitions to include IRE, HFIRE, thermal ablation and/or electrolysis. Zone two would likely represent a combination of anti-inflammatory and proinflammatory cell death mechanisms where close to the electrodes and high intensity electrical fields, the other ablation modalities dependent damage will contribute to necrosis and pyroptosis. During zone two substantially the entire tumor microenvironment is going through non-homogenous cell death mechanisms from center to periphery. The regions of zone two that are undergoing apoptosis and necroptosis may contain a microenvironment to induce an anti-tumor immune response; whereas the rest of zone two is undergoing damage from other ablation modalities which may stop angiogenesis and hence provide more oxygen for the coming infiltrated T cells. However, since the supply of oxygen depends on the number of vasculature and perfusion rate, the tissue with target area undergoes hypoxia. The impact of zone 3 on an extracellular membrane is significant and due to the plasma membrane disruption, the proinflammatory signals are activated and so the innate and adaptive immune responses. This scenario would serve a case where the adaptive immune system is not yet activated and therefore systemic immune response (cold tumors, immunosuppressed) needs to be induced. In one non-limiting example, after a few hours to days, the angiogenesis and vasculogenesis will occur as a natural healing process due to the injury, and therefore there will be enough oxygen supply for the infiltrated cells which can be provided by other adjuvant therapies to the tumor microenvironment.

In one embodiment (not shown), the ablation device described herein comprises a sensor feedback mechanism to better define or identify electrolysis zones (i.e., transition with thermal and IRE/HFIRE) and electrolysis zone. The sensory feedback mechanism is used to monitor for electrolysis factors. Electrolysis factors comprise tissue properties linked to electrolysis (i.e., PH changes). Electrolysis is a chemical ablation mechanism of action, and the extent of ablation is a function of the concentration of the chemical species and the exposure time to such chemicals. The sensory feedback mechanism may comprise a sensor to monitor PH levels or changes, and/or temperature. For example, the electrode may be operatively coupled to the sensory feedback mechanism and is configured to monitoring electrolysis factors. The system can then compare the monitored electrolysis factor information with the normalized current value to provide feedback to a user to identify the point in time in which a target tissue undergoes electrolysis. The monitored electrolysis factor information may also be included in a treatment database to aid in the machine learning aspects of this disclosure (as described in more detail below).

In one embodiment, the device provides intra-treatment feedback in real-time to a clinician regarding which therapy zone the treatment is in as well as to predict progression of the treatment through the therapy zones. FIG. 8 illustrates a plot 870 showing a normalized current trend lines 872, 874, 876, 878 where number of pulses 886 is represented on the X axis and both a change in normalized current 884 and a percentage change in normalized current 882 are on the Y axis. As shown, the normalized current 884 has an initial value of 1.0 for all four trend lines 872, 874, 876, and 878. In one non-limiting example, as pulses are delivered, the normalized current 884 continues to increase for all trend lines 872, 874, 876, and 878 until approximately 120 pulses have been delivered. The transition point 888 from zone one 892 to zone two 894 is defined by a drop in the normalized current 884 value for all trend lines 872, 874, 876, 878. Although the normalized current values and transition point 888 between zones will vary from procedure to procedure, the drop in normalized current is a reliable indicator of a change in the mechanism of cell death. For example, zone one 892 may indicate to the user that cell death of the target tissue is primarily due to irreversible electroporation. Transition 890 to zone two 894 may indicate that mechanism of cell death is transitioning from primarily IRE to another ablation modality (such as thermal ablation). This information can be used by a clinician for several different purposes, including, but not limited to, determining if additional pulses should be delivered, determining the type of cell death occurring in the target area, determining the potential immune response the body may have as a result of additional electrical pulses to be delivered, and/or determining the completeness of the IRE and/or H-FIRE treatments. Plotting of the rate of change in normalized current and/or the percentage change in current, as shown in FIG. 8, may also be used to assist the clinician in identifying the onset of reversible electroporation, irreversible electroporation, thermal ablation, and electrolysis in the target cell zone. For the sake of clarity, FIGS. 8, 9, and 11 do not depict zone zero, however FIG. 23 includes zone zero (as described in more detail below).

FIG. 8 may provide the user with feedback on a planned ablation procedure and where within the ablation procedure the therapy zones might progress based on planned procedure protocol parameters. For example, zone one may comprise a predominantly IRE and/or HFIRE mechanism of action, resulting in a sudden rise in normalized current 827, 874, 876, 878. During use, the user may select various treatment parameters, such as a first parameter 827, a second parameter 874, a third parameter 876, and/or a fourth parameter 878. For example, if user selects 2700V (second parameter 874) the treatment planning plot 870 will display the normalized current and percent change in current for the selected parameter(s) 2700 V, as well as the transition between hypothetical zone one. The user may then decide to select a different treatment parameter based on this information provided by the plot 870. This allows the user to see how various treatment parameters will potentially impact the normalized current, percent change in current and the transition between hypothetical zones. Transition zone 888 suggests to a user a change in electrical properties of the tissue. Zone two is still predominantly IRE and/or HFIRE mechanism of action, but the effect of other ablation modalities (i.e., thermal ablation and/or electrolysis) are not negligible when it comes to cell death mechanisms and consequently triggering different immune reaction. Transition zone 890 shows a transition between zone two and zone three. Within zone three the effect of other ablation modalities (i.e., thermal ablation and/or electrolysis) is a substantial part of the treatment which trigger different cell death pathways and immune response.

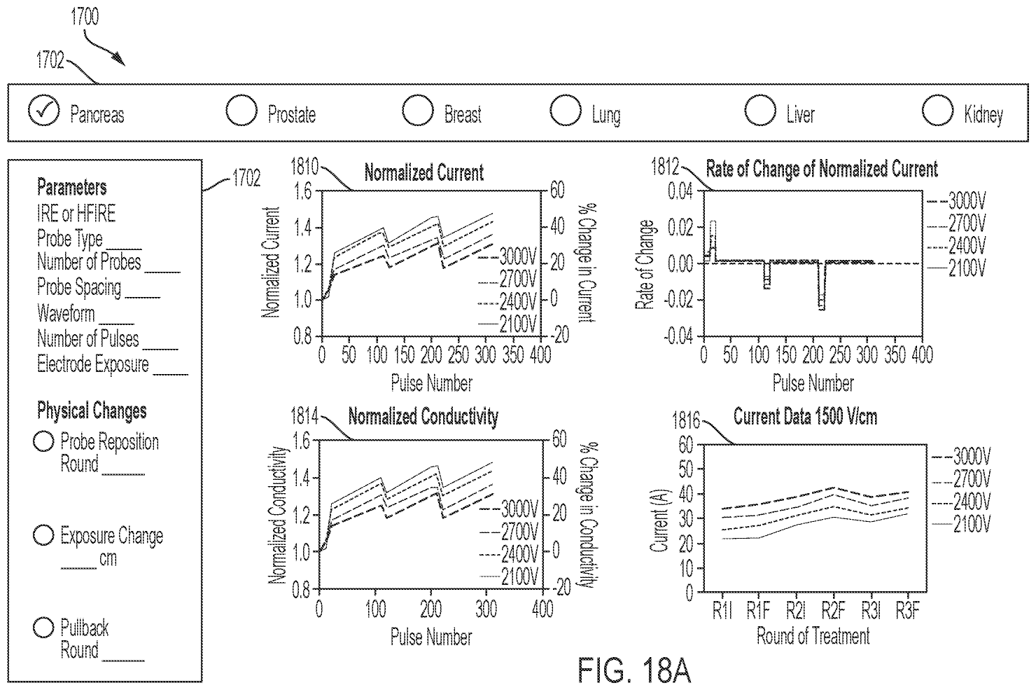

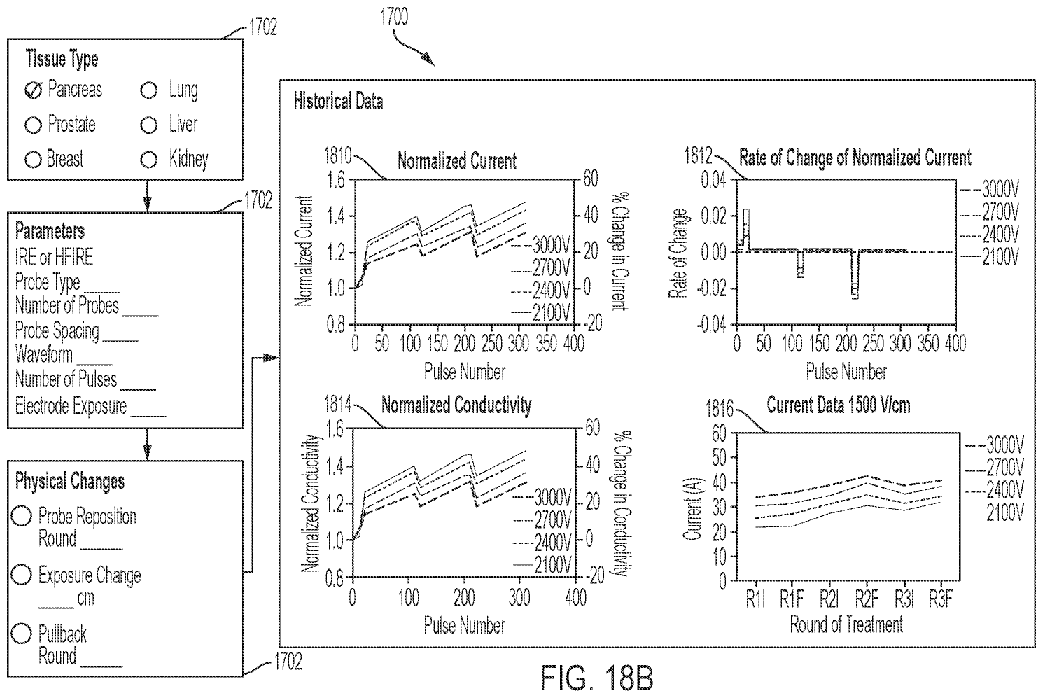

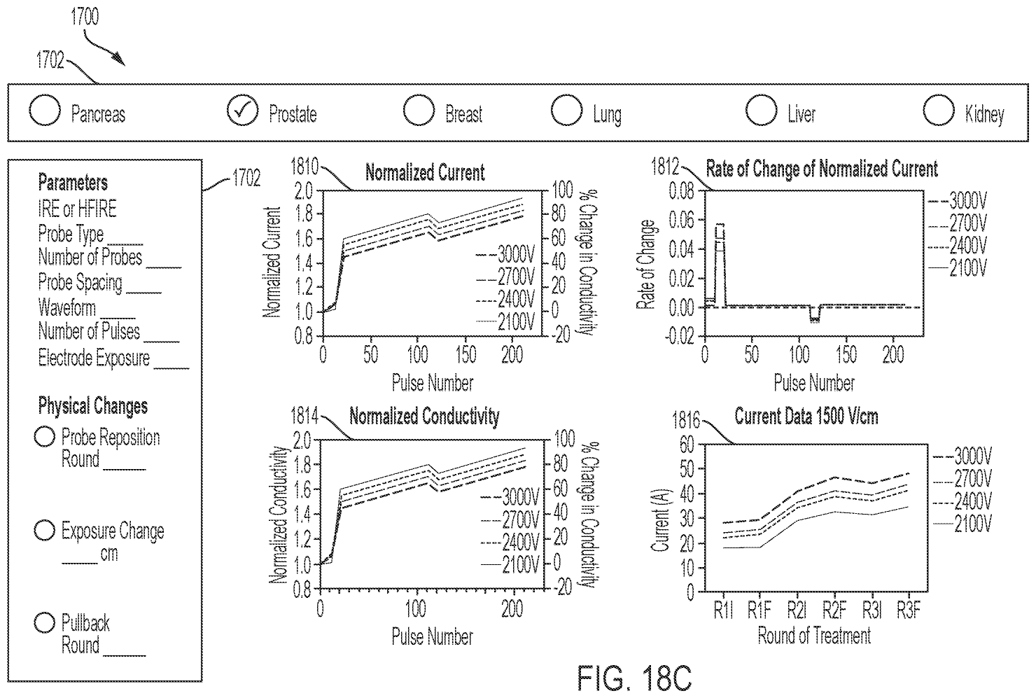

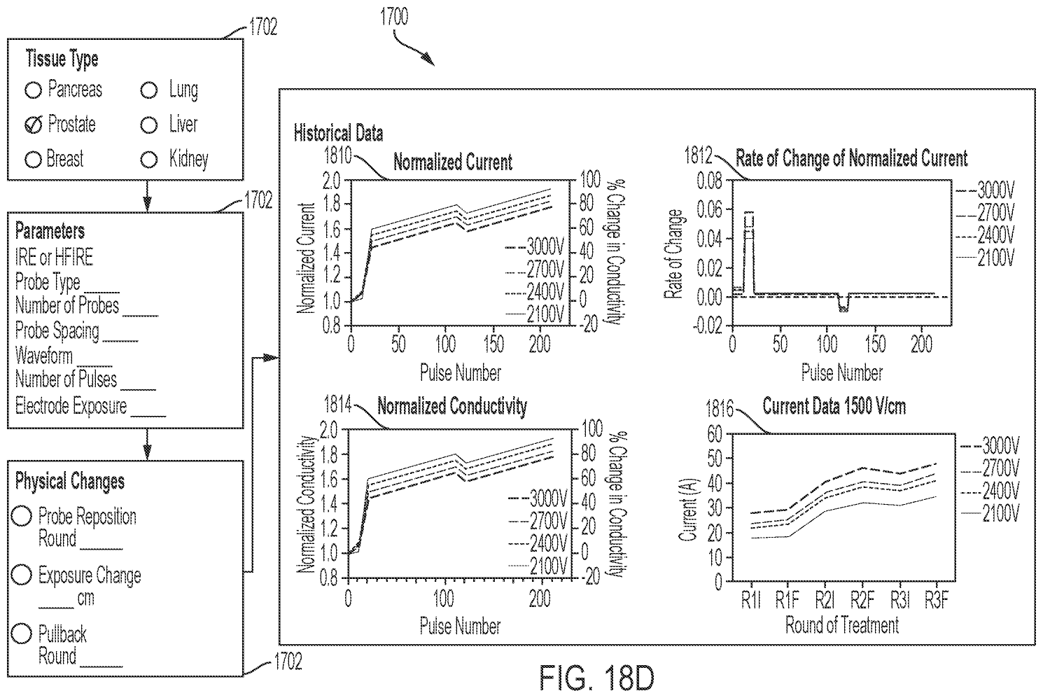

The normalized current plot 870 shown in FIG. 8 also illustrates the maximum Percent Change in Normalized Current (PCNC) that the target tissue reaches in addition to the maximum PCNC achieved as the treatment progresses from one zone to another as described herein. Using the 2100V trend line 878 (which represents the largest change in PCNC depicted in the plot), the PCNC at pulse 120 is approximately 40%, rising to approximately 50% at pulse 220 and returning to a PCNC of approximately 50% at pulse 325. Furthermore, again using the 2100V trend line 878 the PCNC increased 40% within zone one, an incremental increase of 10% within zone two, and no significant incremental increase within zone three, with the total PCNC increases about 50%. Furthermore, at pulse 120, where the electrical energy impact on the target tissue transitions from predominately IRE to other ablation modes, the PCNC is at 40% increase from the baseline. This suggests that the percentage changes from the baseline for electrical conductivity will be limited for transition or thermal zones. When switching to the next probe pair, the ablated tissue will have enough time to get rid of the accumulated heat by conduction or convection. In other words, the rise in current and conductivity within these zones will be diminished after the completion of pulse delivery, solely because these electrical conductivity changes are due to the temperature changes and not due to electroporation (˜2-3% change in electrical conductivity/° C.). Referring to FIG. 18 (B), the Percent Change in Normalized Conductivity (PCNσ) may also be analyzed in a similar manner. Although the maximum of both PCNC and PCNσ values will vary based on the type of tissue being treated, these calculations may be used for pre-treatment planning purposes or to estimate dynamic conductivity of the target tissue in real time.

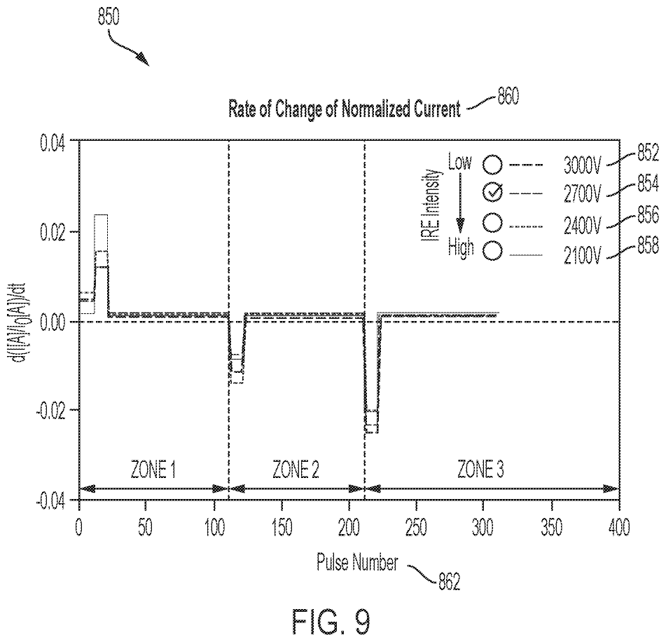

FIG. 9. depicts an example plot 850 in which the user may use a hypothetical normalized current measurement as a parameter for treatment planning. The user may use the data stored in the treatment database (described in detail below) to perform treatment planning. The data to support the treatment planning plot 850 parameters comprise the rate of change of the hypothetical normalized current measurements 860, pulse numbers 862, hypothetical IRE intensity, and hypothetical zone one, hypothetical zone two, and hypothetical zone three may be derived from the treatment database as described in detail below. During use, the user may select a tissue type (not shown) and select between various treatment parameters, such as a first parameter 852, a second parameter 854, a third parameter 856, a fourth parameter 858. For example, if user selects 2700V (second parameter 854) the treatment planning plot 850 will display the rate of change of hypothetical normalized current for 2700 V, the IRE intensity for 2700 V, and the transition between hypothetical zone one, hypothetical zone 2 and hypothetical zone three for 2700 V. The user may then decide to select a different treatment parameter based on the information provided by the plot 850. This allows the user to see how various treatment parameters will potentially impact the rate of change of normalized current, the IRE intensity, and the transition between hypothetical zones.

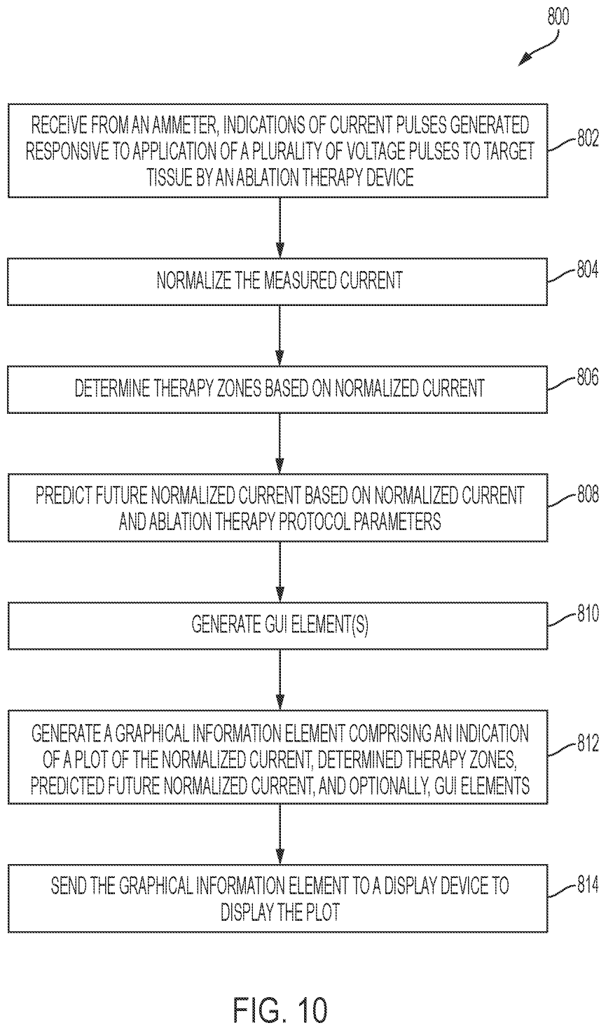

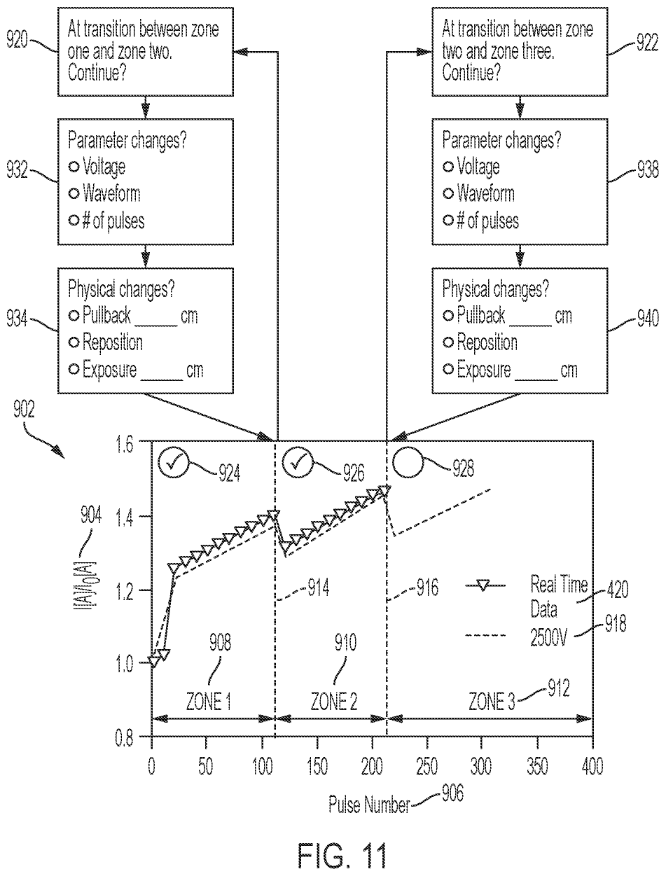

FIG. 10 depicts a routine 800 that may be implemented by an ablation therapy device according to examples of the present disclosure. In some examples, routine 800 can be implemented to generate graphical data for display on display 410. For example, FIG. 11 depicts an example plot 902 that may be generated based on routine 800. The routine 800 of FIG. 10 and plot 902 of FIG. 11 are described together herein.

The system may be capable of automatically stopping or pausing the delivering of electrical pulses upon the completion of a therapy zone. For example, prior to the delivery of electrical pulses, the user may select an option so the voltage source 104 is to stop or pause the delivery of electrical pulses once the normalized current measurement indicates that the treatment has completed a selected therapy zone or a transition between selected therapy zones.

As discussed herein, the primary mechanism(s) of cell death within the target tissue may be classified in zones based on the trend of the normalized current. For example, the first few pulses (e.g., up to first 5 pulses) the cells undergo predominantly reversible electroporation (discussed in more detail below and shown as “zone zero” in FIG. 23). As the normalized current progresses within zone one 908 the mechanism(s) of cell death is predominantly irreversible electroporation. As the normalized current transitions between zone one 908 into zone two 910 and then progresses within zone two 910 the mechanism of cell death predominantly transitions between irreversible electroporation to thermal ablation. As the normalized current transitions between zone two 910 into zone three 912 and then progresses within zone three 912 the mechanism of cell death is predominantly continued thermal ablation and/or electrolysis. Therefore, mechanism(s) of cell death occur at different levels of severity and/or different mechanisms of cellular death as normalized current transitions between zone one 908, zone two 910, and zone three 912. FIG. 11 and FIGS. 18(A)-18(D) are illustrative examples. Furthermore, when the mechanism of action is predominantly irreversible electroporation the cell death is predominantly apoptotic cell death; whereas when the mechanism of action is predominantly thermal ablation the cell death is predominantly necrotic cell death. When the mechanisms of action of cell death comprises different mechanisms of action (i.e., ire, thermal, electrolysis, and/or a transition between these mechanisms of action), multiple cell death pathways (instead of single dominant cell death pathway) are expected. Moreover, tissue will undergo apoptotic cell death in a milder excitation (i.e., irreversible electroporation) mechanism of action (non-inflammatory nor PMPs or DAMPs triggered) and undergo necrosis or pyroptosis when exposed to harsher excitation (i.e., thermal ablation, high dose of radiation, and/or chemo-therapy) which will be proinflammatory with induction of PAMPs and DAMPs.

FIG. 11, illustrates a plot 902 showing a normalized current trend line 421 where number of pulses 906 is represented on the X axis and changes in normalized current 904 the Y axis. As shown, the normalized current 420 has an initial value of 1.0. As pulses are delivered, the normalized current 420 continues to increase until approximately 120 pulses have been delivered. The transition point 914 from zone one 908 to zone two 910 is defined by a drop in the normalized current 420 value. Although the normalized current values and transition point 914 between zones will vary from procedure to procedure, the drop in normalized current is a reliable indicator of a change in the mechanism of cell death. For example, zone one 908 may indicate to the user that cell death of the target tissue is primarily due to irreversible electroporation. Transition to zone two 910 may indicate that mechanism of cell death is transitioning from primarily IRE to another ablation modality. This information can be used by a clinician for several different purposes, including, but not limited to, determining if additional pulses should be delivered, determining the type of cell death occurring in the target area, determining the potential immune response the body may have as a result of additional electrical pulses to be delivered, and/or determining the completeness of the IRE and/or H-FIRE treatments. Also shown in FIGS. 18(A)-18(D), the rate of change of the normalized current (electroporation intensity) initially falls below zero at approximately 120 pulses. The start of zone three of FIG. 11 and the second negative rate of change in FIG. 18 (A)-18(D) both begin after approximately 120 pulses.

As shown in FIG. 11, at the first transition point 914 a series of GUI elements may be displayed on a display unit to prompt user input. For example, GUI elements at first transition point 914 may comprise a query of “At transition between zone one and zone two. Continue?” 920; a query of “Parameter Change?” 932 (i.e., change to voltage, waveform, or number of pulses); a query of “Physical Changes?” 934 (i.e., a pullback length, reposition probes, or exposure length). Also shown at the second transition point 916 a series of GUI elements may be displayed on a display unit to prompt user input. For example, GUI elements at first transition point 916 may comprise a query of “At transition between zone two and zone three. Continue?” 922; a query of “Parameter Change?” 938 (i.e., change to voltage, waveform, or number of pulses); a query of “Physical Changes?” 940 (i.e., a pullback length, reposition probes, or exposure length). These GUI elements are non-limiting examples, and it is within the scope of this disclosure other parameters common for ablation procedures that be included as GUI elements.

A threshold number of pulses that triggers both a zone change in the normalized current trend and decrease in rate of normalized current change is not constant but rather will vary based on intrinsic and extrinsic factors. As an example, if all other extrinsic and intrinsic factors assumed equal, tissue with a low baseline conductivity will require more pulses before transitioning to the next zone than higher conductivity tissue. As another example, a probe pair placed further apart will require more pulses before transitioning to a second zone and a negative rate of normalized current change than probe pairs placed closer together.

Referring back to FIG. 9, at block 802 “receive from the ammeter, indications of current generated responsive to application of a plurality of voltage pulses to the target tissue by an ablation therapy device” indications of current measured at an ammeter can be received. For example, in executing instructions 416 processor(s) 408 can receive measured current 418 from ammeter 406.

At block 804 “normalize the measured current” the measured current can be normalized. For example, in executing instructions 416 processor(s) 408 can normalize the measured current 418 to generate normalized current 420. In some examples, processor(s) 408 can execute instructions to normalize the measured current for voltage. Said differently, processor(s) 408 can execute instructions 416 to normalize measured current 418 to a common reference point, resulting in normalized current 420. Actual normalized current 420 is depicted as normalized current 904 on the Y axis of plot 902 versus pulse number 906, which are depicted on the X axis of plot 902.

At block 806 “determine therapy zones based on normalized current” therapy zones can be based on the normalized current 420. In general, therapy zones and transitions between therapy zones can be defined by a change (e.g., percent increase/decrease, increase/decrease greater than a threshold value, or the like) in the normalized current 420. For example, 902 depicts treatment zone one 908, treatment zone two 910, and treatment zone three 912. Additionally, transition one 914 between zone one 908 and zone two 910 as well as transition two 916 between zone two 910 and zone three 912 are depicted. As a non-limiting example, therapy zone one 908 may comprise a therapy zone in which tissue within the target site being ablated predominantly by irreversible electroporation. Therapy zone two 910 may comprise a therapy zone in which irreversible electroporation and temperature-related cell death mechanisms working together. The increase in normalized current 420 may be caused by a conductive rise due to an increase in the base target tissue temperature. In general, there is ˜2% rise in conductivity of tissue for each degree rise in temperature of ablated tissue. The temperature rise may begin to occur in zone one but is dependent on extrinsic and intrinsic factors. Therapy zone three 912 may comprise a therapy zone in which the tissue within the target site is no longer the predominantly impacted by the irreversible electroporation cell death mechanism due to the fact that most of the electrical conductivity changes in the tissue is resulted from temperature changes (i.e., predominantly thermal ablation and/or electrolysis) and not the IRE effects. With some examples, processor(s) 408 in executing instructions 416 can determine therapy zones and transitions between therapy zones based on the normalized current 420.

At block 808 “predict future normalized current based on normalized current and ablation therapy protocol parameters” future normalized current can be predicted based on actual normalized current 420 and ablation therapy protocol parameters 424. For example, processor(s) 408 in executing instructions 416 can predict future normalized current for the ablation therapy given past normalized current 420 and the ablation therapy protocol parameters 424. With some examples, processor(s) 408 in executing instructions 416 can predict future normalized current based on machine learning models trained on completed ablation therapy protocols. As another example, processor(s) 408 in executing instructions 416 can predict future normalized current based a mathematical relationship between normalized current 420 and ablation therapy protocol parameters 424. Predicted future normalized current 918 is depicted in plot 902. In particular, plot 902 depicts both actual normalized current 420 (e.g., normalized current for voltage pulses actually applied during treatment) and predicted future normalized current 918 (e.g., normalized current for voltage pulses not yet applied but scheduled based on ablation therapy protocol parameters 424, or the like). Accordingly, plot 902 provides an intra-treatment picture of where within the therapy zones the treatment therapy is and also where within the therapy zones the treatment therapy might progress based on actual protocol parameters 424.

At block 810 “generate GUI element(s)” graphical user interface (GUI) elements can be generated. For example, in executing instructions 416 processor(s) 408 can generate graphical data (e.g., display frames, or the like) including indications of GUI element 920, GUI element 922, GUI input element 924, GUI input element 926, GUI input element 928, GUI input element 930, GUI input element 932, GUI input element 934, GUI input element 936, GUI input element 938, GUI input element 940. With some examples, GUI elements can include an indication of where within the therapy zones the ablation treatment is, an indication that the ablation therapy treatment is approaching a transition between zones (e.g., GUI element 920, GUI element 922, or the like), a query if entering a transition zone and want to continue, a query if there is a parameter change (e.g., voltage, waveform, number of pulses), a query if there are any physical changes to probes (e.g., pull back length, reposition probes, change electrode exposure length), a query to continue the ablation therapy treatment, or said differently continue application of voltage pulses (e.g., GUI input element 924, GUI input element 926, GUI input element 928, or the like).

At block 812 “generate a graphical information element comprising an indication of a plot of the normalized current, determined treatment zones, predicted future normalized current, and optionally, GUI elements” graphical data (e.g., display frames, or the like) representing plot 902 can be generated comprising indications of normalized current 420, predicted future normalized current 918, treatment zone one 908, treatment zone two 910, treatment zone three 912, predicted future normalized current 918, GUI element 920, GUI element 922, GUI input element 924, GUI input element 926, and/or GUI input element 928. The graphic data can be stored in memory 414 as graphical information element 426.

At block 814 “send the graphical information element to a display device to display the plot” the ablation therapy device can send the graphical information element 426 to display 410 to display the plot indicated by the graphical information element 426. For example, in executing instructions 416 processor(s) 408 can send the graphical information element 426 to display 410 and display 410 can display the plot indicated by the graphical information element 426.

In some examples, routine 800 can be repeated such that display 410 can be updated with indications of plot 902 with updated information (e.g., updated normalized current 420, updated predicted future normalized current 918, updated GUI elements, or the like) as the treatment progresses.

Further, routine 800 can be repeated individually for each pair of probes or collectively for all probe pairs. For example, routine 800 can be implemented such that a plot depicting normalized current from one pair of probes can be generated at block 808 and can be repeated such that another plot depicting normalized current from another pair of probes can be generated at block 808. In some examples, both plots can be displayed on display 410. In other examples, a single plot depicting normalized current from multiple pairs of probes can be generated.

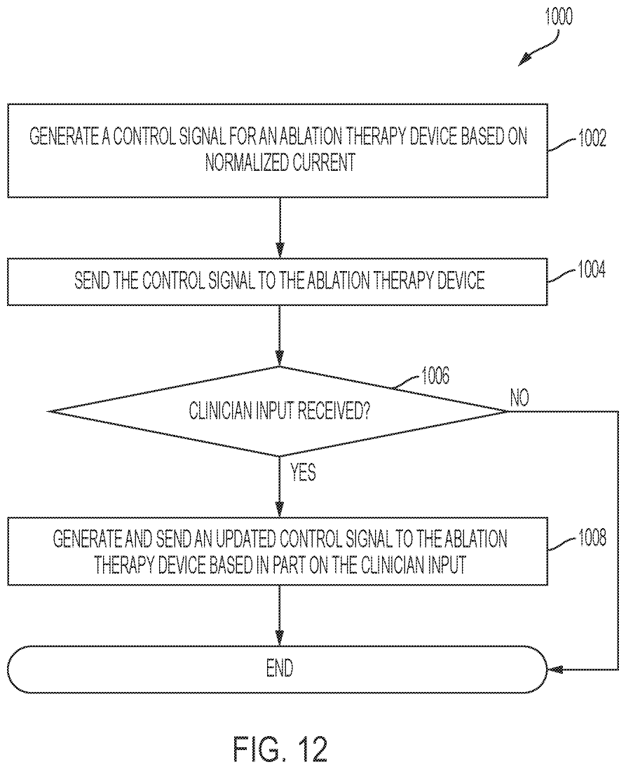

FIG. 12 depicts a routine 1000 that may be implemented by an ablation therapy device according to examples of the present disclosure. In some examples, routine 1000 can be implemented to control the ablation therapy device based on normalized current. At block 1002 “generate a control signal for an ablation therapy device based on normalized current” control signal is generated for the ablation therapy device based on normalized current. For example, processor(s) 408 in executing instructions 416 can generate control signal 422. As a specific example, where normalized current 420 indicates that the ablation therapy is at a transition between treatment zones (e.g., at transition one 914 as indicated by GUI element 920 of FIG. 11, or the like), processor(s) 408 in executing instructions 416 can generate a control signal including an indication for voltage generator 404 to pause generation of voltage pulses. As another example where the normalized current 420 increases more than a threshold value (e.g., more than a percentage increase, more than a magnitude of increase, or the like), processor(s) 408 in executing instructions 416 can generate a control signal including an indication for voltage generator 404 to pause generation of voltage pulses.

At block 1004 “send the control signal to the ablation therapy device” control signal 422 can be sent to the ablation therapy device 400. For example, controller 106 can send the control signal 422 to voltage source 104. At decision block 1006 “clinician input received?” a determination can be made whether clinician input 428 is received. For example, responsive to a GUI input element (e.g., GUI input element 924, GUI input element 926, GUI input element 928, or the like), processor(s) 408 in executing instructions 416 can receive input from a clinician. As a specific example, a clinician can use I/O devices 412 to provide a response to the GUI element and/or GUI input element and processor(s) 408 can receive the response at decision block 1006. From decision block 1006, routine 1000 can continue to either block 1008 or can end. For example, routine 1000 can proceed from decision block 1006 to block 1008 based on a determination that clinician input was received while routine 1000 can end based on a determination that clinician input was not received.

At block 1008 “generate and send an updated control signal to the ablation therapy device based in part on the clinician input” an updated control signal can be generated and sent to the ablation therapy device based on the clinician input 428. For example, where the clinician input is to continue the ablation therapy treatment, the updated control signal 422 can include an indication to resume generating and applying voltage pulses to the target tissue 112. In other examples, the clinician input can be an indication to change protocol parameters 424 (e.g., change voltage or other parameters, or the like).

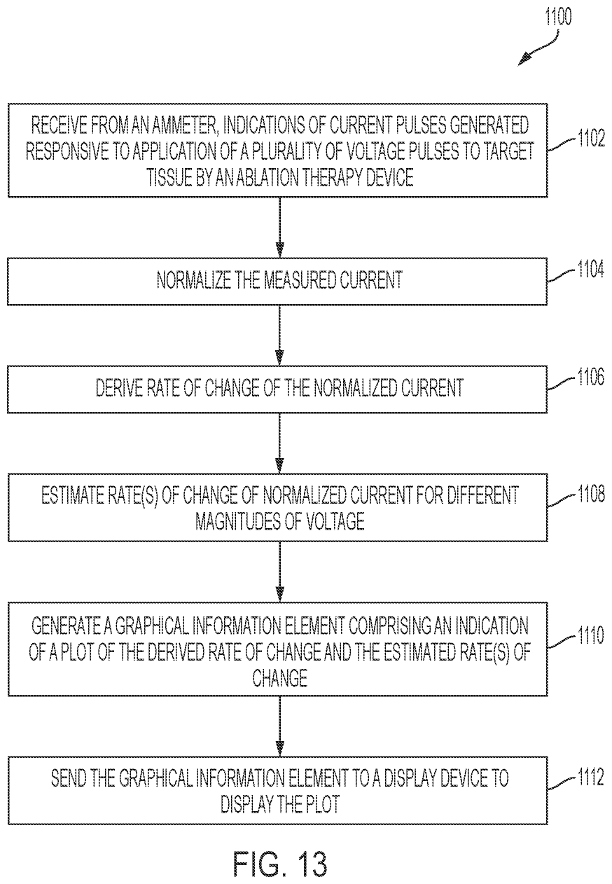

FIG. 13 depicts a routine 1100 that may be implemented by an ablation therapy device according to examples of the present disclosure. The routine 1100 of FIG. 11 and plots of FIG. 18(A)-18(D) are described together herein. In some examples, routine 1100 can be implemented to generate graphical data for display on display 410. For example, FIGS. 18(A)-18(D) depicts example plots that may be generated based on routine 1100. In particular, FIGS. 18(A)-18(D) shows plots depicting rate of change of normalized current and estimated rate of change of normalized current for a probe pair over a number of voltage pulses of an ablation therapy treatment.

The rate of change of normalized current can be used to indicate an “intensity” of an IRE or H-FIRE procedure. The intensity can be defined in the first derivative of the normalized current as a constant value either positive, zero, or negative. When the intensity is positive, IRE intensity is higher and/or has a stronger effect on the target tissue. In the normalized current graph as shown in FIG. 11, if the slope of real-time data of normalized current is positive, the IRE intensity is stronger as compared with the slope of real-time data of normalized current is negative (as seen during the transition periods 914, 916). Therefore, when the real-time data of normalized current has a steeper slope (ex: between pulses 3-20) the IRE has a stronger intensity on target tissue as compared to when the real-time data of normalized current has a shallow slope (ex: between pulses 21-125). In other words, the intensity of the IRE and/or H-FIRE procedure may include the completeness of irreversible electroporation of tissue within a target site, the type of cell death of the tissue, and/or the potential effects the ablation may have on an immune response by the body. In general, a more positive rate of change of the normalized current (e.g., positive first derivative of the normalized current) indicates a more intense IRE or H-FIRE procedure. The present disclosure can be implemented to determine an intensity of an IRE or H-FIRE procedure and compare the determined intensity with estimated or predicted intensities for the procedure at different voltages. With some examples, routine 1100 can generate graphical information element including indication of a plots 1810, 1812, 1814, 1816 providing a graphical representation of the intensity of an IRE and/or H-FIRE procedure for various voltage settings. At block 1102 “receive from the ammeter, indications of current generated responsive to application of a plurality of voltage pulses to the target tissue by an ablation therapy device” indications of current measured at an ammeter can be received. For example, in executing instructions 416 processor(s) 408 can receive measured current 418 from ammeter 406.

At block 1104 “normalize the measured current” the measured current can be normalized. For example, in executing instructions 416 processor(s) 408 can normalize the measured current 418 to generated normalized current 420. In some examples, processor(s) 408 can execute instructions to normalize the measured current for voltage. Said differently, processor(s) 408 can execute instructions 416 to normalize measured current 418 to a common reference point, resulting in normalized current 420.

At block 1106 “derive rate of change of normalized current” a rate of change of normalized current 420 can be derived. For example, processor(s) 408 in executing instructions 416 can determine an actual rate of change of the normalized current 420 using Equation 5 described herein.

At block 1108 “estimate rate(s) of change of normalized current for different magnitudes of voltage” rates of change of normalized current can be estimated for different magnitudes of voltage with which an ablation therapy could be applied. For example, processor(s) 408 in executing instructions 416 can estimate a rate of change of normalized current for other ablation therapy procedure voltages (e.g., voltage magnitudes different than the current magnitude, or the like). For example, processor(s) 408, in executing instructions 416, can determine and display a plot comprising an estimated rate of change of normalized conductivity versus pulse numbers (not shown), and/or normalized conductivity versus voltage gradient (not shown).

At block 1110 “generate a graphical information element comprising an indication of a plot of the derived rate of change and the estimated rate(s) of change” graphical data (e.g., display frames, or the like) representing plots can be generated comprising indications of change in normalized current 1810, the rate of change of normalized current 1812, change in percentage of change in conductivity 1814, and indication of combined treatment data from multiple physicians 1816. The graphic data can be stored in memory 414 as graphical information element 426.

At block 1112 “send the graphical information element to a display device to display the plot” the ablation therapy device can send the graphical information element 426 to display 410 to display the plot indicated by the graphical information element 426. For example, in executing instructions 416 processor(s) 408 can send the graphical information element 426 to display 410 and display 410 can display the plot indicated by the graphical information element 426 (e.g., plots 1810, 1812, 1814, 1816).

In some examples, routine 1100 can be repeated such that display 410 can be updated with indications of plots 1810, 1812, 1814, 1816 with updated information as the treatment progresses.

Further, routine 1100 can be repeated individually for each pair of probes or collectively for all probe pairs. For example, routine 1100 can be implemented such that a plot depicting normalized current from one pair of probes can be generated at block 1108 and can be repeated such that another plot depicting normalized current from another pair of probes can be generated at block 1108. In some examples, both plots can be displayed on display 410. In other examples, a single plot depicting actual rate of change from multiple pairs of electrodes (not shown) can be generated.

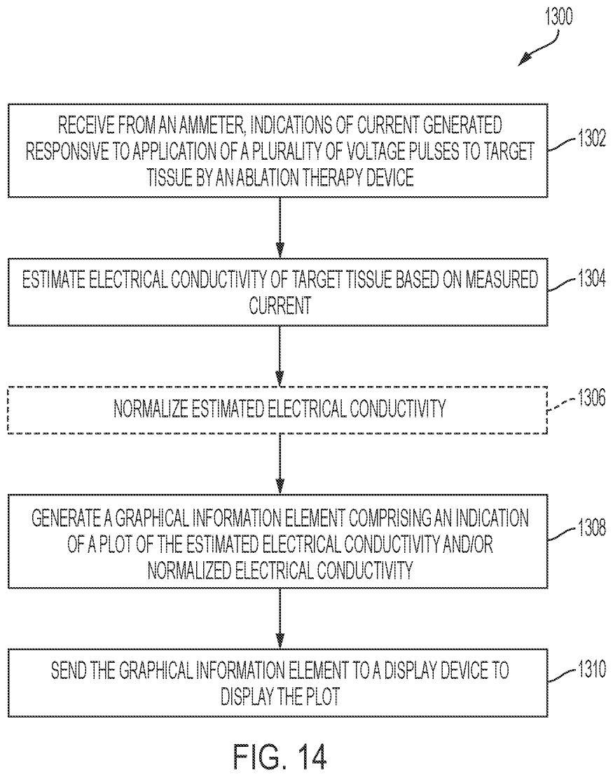

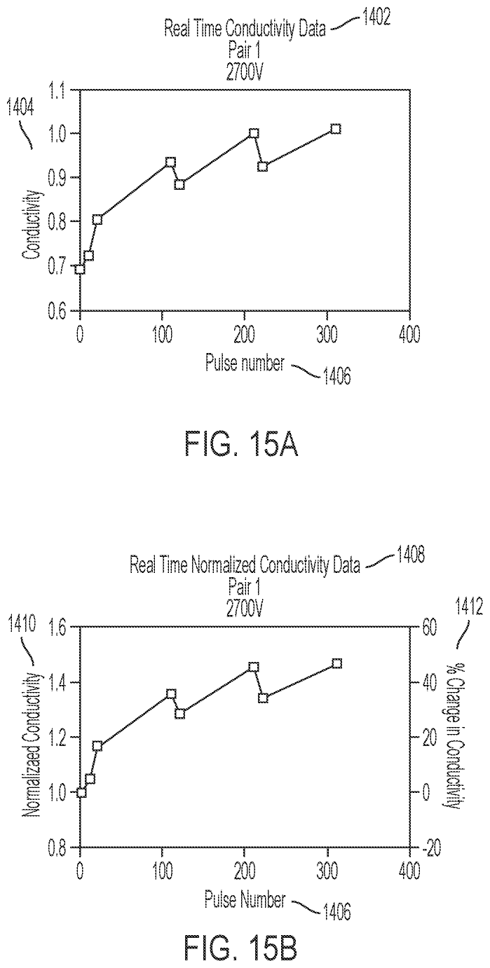

FIG. 14 depicts a routine 1300 that may be implemented by an ablation therapy device according to examples of the present disclosure. In some examples, routine 1300 can be implemented to generate graphical data for display on display 410. For example, FIG. 15(A) and FIG. 15(B) depict examples of plot 1402 and plot 1408, respectively, which may be generated on routine 1300. The routine 1300 of FIG. 14, plot 1402 of FIG. 15(A), and plot 1408 of FIG. 15(B) are described together herein.

At block 1302 “receive from the ammeter, indications of current pulses generated responsive to application of a plurality of voltage pulses to the target tissue by an ablation therapy device” indications of current measured at an ammeter can be received. For example, in executing instructions 416 processor(s) 408 can receive measured current 418 from ammeter 406.

At block 1304 “estimate electrical conductivity of target tissue based on measured current” electrical conductivity of target tissue can be estimated (e.g., derived, or the like) based on measured current 418. For example, processor(s) 408 in executing instructions 416 can estimate electrical conductivity of target tissue 112 given measured current 418. It is to be appreciated that electrical conductivity and measured current 418 have close relationship and that normalized values of both are substantially the same if the shape factor is assumed to be constant throughout the ablation therapy procedure. Equation 1 and 2 detailed above illustrate the relationship between current (I), shape factor (S), and electrical conductivity (a). The shape factor, C, defines probe-specific characteristics which impact tissue response and the resulting current measurement. Probe characteristics include probe dimensions, electrode dimensions and distance between probes. As an example, the following Equation 6 can be used to represent probe dimensional when using two or more cylindrical probes placed in parallel relationship in the target tissue. Where s=shape factor; L=electrode exposure length; D1=diameter of probe #1; D2=diameter of probe #2; z=distance between probe #1 and probe #2. Note, it is within the conception of this disclosure to use more than two probes, and Equation 6 would be adjusted to reflect a total probe count.

$$S = \frac{2 π L}{{cosh}^{- 1} ( \frac{4 {z}^{2} - {D}_{1}^{2} - {D}_{2}^{2}}{2 {D}_{1} {D}_{2}} )} \qquad Equation 6$$

At block 1306 “normalize estimated electrical conductivity” the estimated electrical conductivity can be normalized. For example, processor(s) 408 in executing instructions 416 can normalize the estimated electrical conductivity derived at block 1304 to a common reference point. Said differently, processor(s) 408 in executing instructions 416 can normalize estimated electrical conductivity for applied voltage.

At block 1308 “generate a graphical information element comprising an indication of a plot of the estimated electrical conductivity and/or the normalized estimated electrical conductivity” a graphical data (e.g., display frames, or the like) representing plot 1402 and/or plot 1408 can be generated. For example, processor(s) 408 in executing instructions 416 can generate graphical information element 426 comprising an indication of plot 1402 where estimated electrical conductivity 1404 is depicted on the Y axis and voltage pulses 1406 on the X axis. Alternatively, or additionally, processor(s) 408 in executing instructions 416 can generate graphical information element 426 comprising an indication of plot 1408 where normalized electrical conductivity 1410 is depicted on the Y axis and voltage pulses 1406 on the X axis. Further, as depicted in plot 1408, the percentage change in normalized electrical conductivity 1412 is depicted in plot 1408. The graphic data can be stored in memory 414 as graphical information element 426.

At block 1310 “send the graphical information element to a display device to display the plot” the ablation therapy device can send the graphical information element 426 to display 410 to display the plot indicated by the graphical information element 426. For example, in executing instructions 416 processor(s) 408 can send the graphical information element 426 to display 410 and display 410 can display the plot indicated by the graphical information element 426 (e.g., plot 1402, plot 1408, or the like).

In some examples, routine 1300 can be repeated such that display 410 can be updated with indications of updated plot 1402, updated plot 1408 to provide intra-treatment indications of estimated electrical conductivity and/or normalized electrical conductivity as the treatment progresses. Further, routine 1300 can be repeated individually for each pair of probes or collectively for all probe pairs.

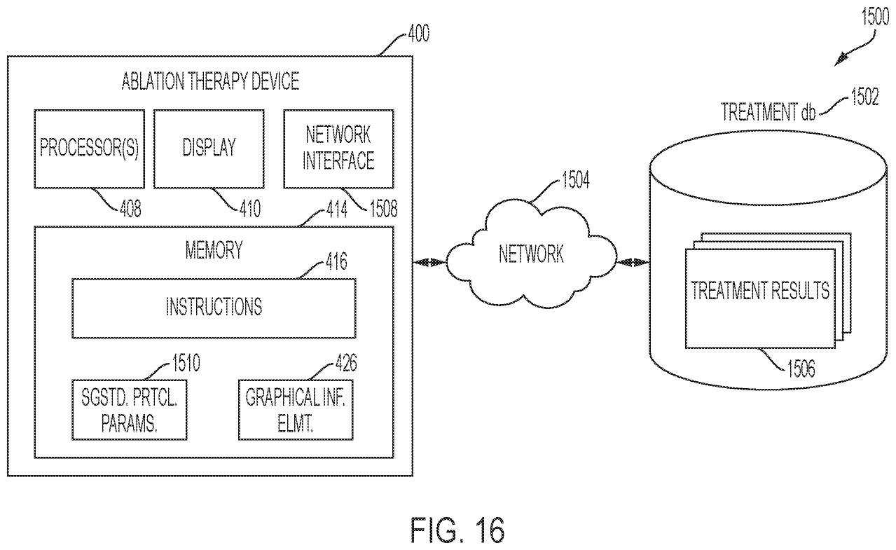

FIG. 16 illustrates an ablation therapy system 1500. In some examples, ablation therapy system 1500 can be implemented to include ablation therapy device 400 of FIG. 4. Although ablation therapy system 1500 is described with respect to ablation therapy device 400, other ablation therapy devices could be implemented in ablation therapy system 1500. Further, it is noted that only a part of ablation therapy device 400 is depicted for brevity.

Ablation therapy system 1500 includes ablation therapy device 400 communicatively coupled to treatment database 1502 via network 1504. In some examples, network 1504 can include the Internet, a local area network, or a wide area network. In some examples, network 1504 can be a private network, such as, for example accessible via virtual private networking (VPN) and/or otherwise credentialed access to protect information exchanged via network 1504. In some examples, network 1504 can be provided by a clinic, a hospital, a research facility, a university, or the like. Access to the network 1504 can be facilitated by a number of computing communication technologies and can include wired (e.g., Ethernet, or the like) or wireless (e.g., Wi-Fi, 4G, 5G, or the like) communication protocols.

Treatment database 1502 can be any of a variety of database structures. In some examples, treatment database 1502 can be provided by a cloud computing environment, such as, a cloud data storage provided. With other examples, treatment database 1502 can be provided by a server, a workstation, a cloud computing service, a virtually hosted computing device, a container computing device, or the like. Treatment database 1502 can store indications of treatment results 1506. In particular, treatment database 1502 can store indications of prior ablation therapy treatments such as, protocol parameters associated with the treatment (e.g., voltage, probe pairs, probe pair placement, voltage pulse details, rounds, etc.), measured current, normalized current, tissue conductivity, normalized tissue conductivity, survivability data (e.g., 1 year survivability statistics, 5 year survivability statistics, etc.), pre and post therapy imaging of the target tissue, or other information related to ablation therapy treatments, including but not limited to target tissue type, disease type, disease state, prior treatments performed, and/or tumor size. The treatment results 1506 can be entered and/or uploaded onto the treatment database 1502 manually by the user, uploading a series of previous treatment results 1506 using an external memory device, upload treatment results 1506 stored on a cloud computing environment or other local network.

During operation, ablation therapy device 400 can operate to access treatment database 1502 to receive treatment results 1506 or to add to treatment results 1506. This is described in greater detail below. However, it is noted that the present disclosure provides reasons for such a database. More specifically, as noted conventionally, data related to one ablation therapy treatment cannot easily be compared to data from another ablation therapy treatment. Said differently, current measured during one ablation therapy treatment cannot easily be compared to current measured during another ablation therapy treatment. However, the present disclosure provides to normalize current to a common reference point such that current from one ablation therapy treatment can more easily be compared to current from another ablation therapy treatment. Thus, clinics and clinicians can contribute to treatment database 1502 to build a bank of treatments with which protocol parameters for future treatments may be based.

In addition to the components detailed elsewhere herein, ablation therapy device 400 can include a network interface 1508. Ablation therapy device 400 can send and receive data (e.g., information elements, data structures, or the like) to/from treatment database 1502 via network 1504 with network interface 1508. For example, network interface 1508 can format data for transmission over network 1504 via a communication protocol or can decode data transmitted over network 1504 via the communication protocol.

Further, ablation therapy device 400 can determine suggested protocol parameters 1510 and generate graphical information element 426 based on treatment results 1506. This and other examples of the disclosure are described in greater detail below.

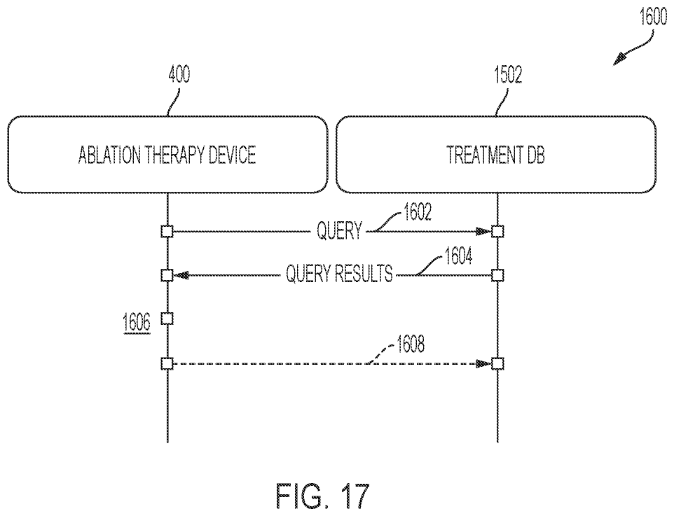

FIG. 17 illustrates technique 1600 detailing operations for ablation therapy device 400 and/or treatment database 1502 according to examples of the present disclosure. In technique 1600, at operation 1602, ablation therapy device 400 can send a query to treatment database 1502. Likewise, at operation 1602 treatment database 1502 can receive a query from ablation therapy device 400. For example, processor(s) 408 in executing instructions 416 can generate a query for treatment database 1502 and can send the query to treatment database 1502 via network interface 1508 and network 1504. As a specific example, the query can include a request to provide treatment results 1506 for a particular target tissue 112 (e.g., pancreas, prostrate, breast, lung, liver, kidney, or the like). With some examples, ablation therapy device 400 can generate query based on input received from a clinician. For example, processor(s) 408 in executing instructions 416 can receive input from a clinician indicating a type of target tissue 112. At operation 1604, treatment database 1502 can send query results to ablation therapy device 400. Likewise, at operation 1604, ablation therapy device 400 can receive query results from treatment database 1502. For example, processor(s) 408 in executing instructions 416 can receive results to a query (e.g., query send at operation 1602, or the like) from treatment database 1502.

At operation 1606, ablation therapy device 400 can generate graphical information element 426 including indications of treatment results 1506 and/or suggested protocol parameters 1510. Examples of this are explained in greater detail below with respect to FIG. 17. However, in general, processor(s) 408 in executing instructions 416 can generate a graphical display (e.g., a plot, multiple plots, or the like) including indications of treatment results 1506. As another example, processor(s) 408 in executing instructions 416 can generate suggested protocol parameters 1510 and a graphical display including indications of suggested protocol parameters 1510. With some examples, suggested protocol parameters 1510 can be generated based on treatment results 1506.

At operation 1608, ablation therapy device 400 can send data including indications of an ablation therapy to treatment database 1502. Likewise, at operation 1608 treatment database 1502 can receive a data from ablation therapy device 400 including indication of an ablation therapy treatment. Furthermore, treatment database 1502 can add the received ablation therapy to treatment results 1506. With some examples, information communicated to 1502 at operation 1608 can include normalized current, estimated electrical conductivity, pre- and post-imaging analysis, survivability results, immune response summary, information about other treatments (e.g., chemo, radiation, thermal ablation, or the like), etc. In some examples, the treatment database 1502 can be updated with patient survivability data (e.g., 1 year survivability statistics, 5-year survivability statistics, etc.) for an extended time post ablation procedure. The survivability data may be accessed and inputted into the treatment database 1502 post treatment procedure by different sources. For example, the survivability data may be manually entered by the same physician performing the initial ablation treatment, manually entered by a different physician who is currently treating the same patient or entered into the treatment database 1502 using data pulled from a patient's electrical medical records.

FIGS. 18(A)-18(D) illustrate graphical display 1700 that can be generated by an ablation therapy device and presented on a display for a clinician. For example, ablation therapy device 400 can generate graphical display 1700 and present on display 410. As a specific example, processor(s) 408 in executing instructions 416 can generate display data (e.g., display frames, or the like) comprising an indication of treatment results 1506 and/or suggested protocol parameters 1510. For example, ablation therapy device 400 can generate tissue selection GUI input elements 1702. For example, input elements may comprise any of the following: tissue type (i.e., pancreas, heart, prostate, breast, lung, liver, kidney); treatment parameters (i.e., RE, IRE or H-FIRE, probe type, number of probes, probe spacing, waveform parameters, number of pulses, electrode exposure length, treatment zone size, margin size); and/or physical change inputs (i.e., probe reposition, electrode exposure length change, or pull back length). Ablation therapy device 400 can receive input from a clinician (e.g., clinician input 428, or the like) regarding type of target tissue 112 via tissue selection GUI input elements 1702. Further, ablation therapy device 400 can generate graphical display elements including one or more plots or suggestions for an ablation therapy treatment for the type of target tissue 112. For example, a plot 1810 comprising indication of normalized current from treatment results 1506 having a same or similar target tissue type can generated and included in graphical display 1700. As another example, a plot 1812 comprising indication of a rate of change of normalized current from treatment results 1506 having a same or similar target tissue type can be generated and included in graphical display 1700. In yet another example, a plot 1814 comprising indication of a normalized tissue conductivity from treatment results 1506 having a same or similar target tissue type can be generated and included in graphical display 1700. With yet another example, a plot 1816 comprising indication of combined treatment data from multiple physicians (e.g., clinicians, or the like) in treatment results 1506 having a same or similar target tissue type can be generated and included in graphical display 1700. As another example, the parameter settings (extrinsic) can be also put in the matrix of information. In general, any parameters that is listed in Equation 1 can be part of this matrix of information. Some text in FIGS. 18(A)-18(D) (Rate of Change of Normalized Conductivity vs. Pulse number, and Normalized Conductivity vs. Voltage Gradient (v/cm)) describe potential future plots or charts that may not be shown, including but not limited to, other extrinsic factors including shape factor, electrode length, distance between electrodes/probes.

In some examples, as depicted, graphical display 1700 can include multiple plots 1810, 1812, 1814, 1816. In other examples (not shown), graphical display 1700 can include a single plot. With still other examples (not shown), graphical display 1700 could include actual suggested parameters (e.g., 4 rounds of 60 pulses each at 2100 volts, or the like) for an ablation therapy treatment for the same or similar target tissue type. With some examples (not shown), the suggested protocol parameters 1510 can be generated based on treatment results 1506 having the highest survivability rates. In other examples (not shown), the suggested protocol parameters 1510 can be generated based on treatment results 1506 where the treatment concluded in a desired treatment zone.

FIGS. 18(C)-18(D) illustrates a graphical display 1700 that can be generated by an ablation therapy device and presented on a display for a clinician. For these examples, ablation therapy device 400 received an input from a clinician (e.g., clinician input 428, or the like) regarding a different type of target tissue 112, as compared to FIGS. 18A-18 B, via tissue selection GUI input elements 1702. In this example user selected a target tissue 112 of prostate tissue. The data used to generate plots 1810, 1812, 1814, 1816 reflect this change in tissue type 112 and can be used to depict the same type of information to user as described above.

FIGS. 18 B and 18D illustrate one example of the flow (represented by arrows) of a user selection of GUI input elements 1702. For example, user may first select a tissue type (including but not limited to pancreas; prostate; breast; lung; liver; and/or kidney), then various treatment parameters (including but not limited to IRE or HFIRE; probe type, number of probes, probe spacing, waveform, number of pulses, and/or electrode exposure), and then physical changes that may occur during a typical procedure (including but not limited to probe reposition round; exposure change; and/or pull back round).

In one example, normalized current and/or normalized conductivity plots depicted in FIGS. 18(A)-18(D) may include, but not limited to, providing the user with the following (i) recommended voltage setting for a procedure; (ii) an expected number of pulses before transition between zones one and two and/or between zones two and three; (iii) the onset and/or transition between the difference mechanism(s) of cell death (i.e., transition between irreversible electroporation, thermal ablation, and/or electrolysis); and/or (iv) when to stop treatment based on a desired outcome. Furthermore, plot indicating the rate of change of normalized current can be used to select treatment options/parameters based on a desired intensity level of irreversible electroporation.

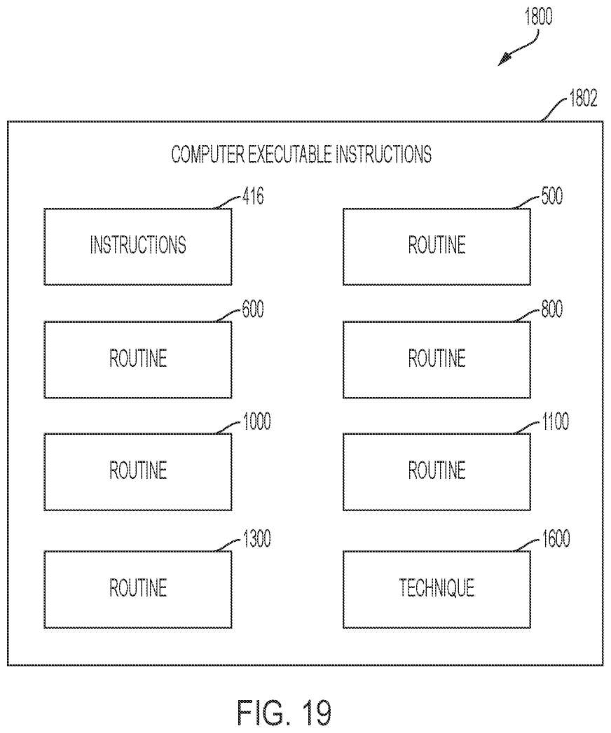

FIG. 19 illustrates computer-readable storage medium 1800. Computer-readable storage medium 1800 may comprise any non-transitory computer-readable storage medium or machine-readable storage medium, such as an optical, magnetic or semiconductor storage medium. In various embodiments, computer-readable storage medium 1800 may comprise an article of manufacture. In some embodiments, 1800 may store computer executable instructions 1802 with which circuitry (e.g., processor(s) 408, or the like) can execute. For example, computer executable instructions 1802 can include instructions to implement operations described with respect to instructions 416, routine 500, routine 600, routine 800, routine 1000, routine 1100, routine 1300, and/or technique 1600. Examples of computer-readable storage medium 1800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1802 may include any suitable type of code, such as source code, compiled code, interpreted code, executable code, static code, dynamic code, object-oriented code, visual code, and the like.

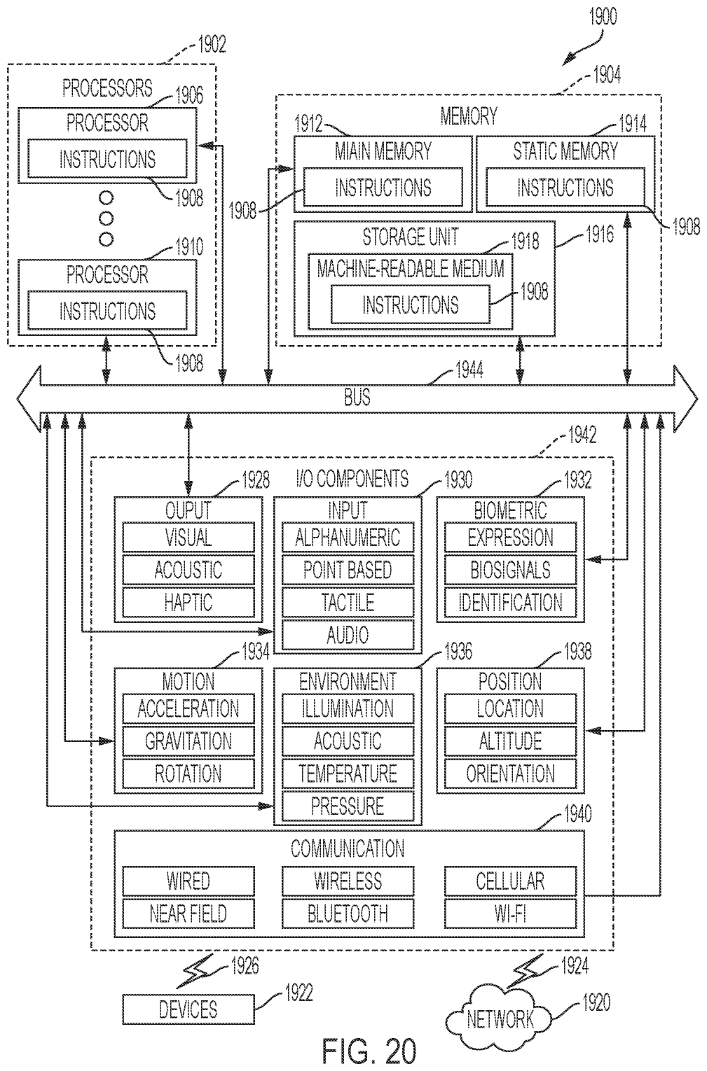

FIG. 20 illustrates a diagrammatic representation of a machine 1900 in the form of a computer system within which a set of instructions may be executed for causing the machine to perform any one or more of the methodologies discussed herein. More specifically, FIG. 20 shows a diagrammatic representation of the machine 1900 in the example form of a computer system, within which instructions 1908 (e.g., software, a program, an application, an applet, an app, or other executable code) for causing the machine 1900 to perform any one or more of the methodologies discussed herein may be executed. For example, the instructions 1908 may cause the machine 1900 to execute instructions 416 of FIG. 4, routine 500 of FIG. 5, routine 600 of FIG. 6, routine 800 of FIG. 10, routine 1100 of FIG. 13, routine 1300 of FIG. 14, technique 1600 of FIG. 17, or the like. More generally, the instructions 1908 may cause the machine 1900 to normalize current from an ablation therapy treatment, generate graphical data for presentation on a display to provide intra-treatment information to a clinician, or interact with a database of treatment results.

The instructions 1908 transform the general, non-programmed machine 1900 into a particular machine 1900 programmed to carry out the described and illustrated functions in a specific manner. In alternative embodiments, the machine 1900 operates as a standalone device or may be coupled (e.g., networked) to other machines. In a networked deployment, the machine 1900 may operate in the capacity of a server machine or a client machine in a server-client network environment, or as a peer machine in a peer-to-peer (or distributed) network environment. The machine 1900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1908, sequentially or otherwise, that specify actions to be taken by the machine 1900. Further, while only a single machine 1900 is illustrated, the term “machine” shall also be taken to include a collection of machines 200 that individually or jointly execute the instructions 1908 to perform any one or more of the methodologies discussed herein.

The machine 1900 may include processors 1902, memory 1904, and I/O components 1942, which may be configured to communicate with each other such as via a bus 1944. In an example embodiment, the processors 1902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1906 and a processor 1910 that may execute the instructions 1908. The term “processor” is intended to include multi-core processors that may comprise two or more independent processors (sometimes referred to as “cores”) that may execute instructions contemporaneously. Although FIG. 19 shows multiple processors 1902, the machine 1900 may include a single processor with a single core, a single processor with multiple cores (e.g., a multi-core processor), multiple processors with a single core, multiple processors with multiples cores, or any combination thereof.

The memory 1904 may include a main memory 1912, a static memory 1914, and a storage unit 1916, both accessible to the processors 1902 such as via the bus 1944. The main memory 1904, the static memory 1914, and storage unit 1916 store the instructions 1908 embodying any one or more of the methodologies or functions described herein. The instructions 1908 may also reside, completely or partially, within the main memory 1912, within the static memory 1914, within machine-readable medium 1918 within the storage unit 1916, within at least one of the processors 1902 (e.g., within the processor's cache memory), or any suitable combination thereof, during execution thereof by the machine 1900.

The I/O components 1942 may include a wide variety of components to receive input, provide output, produce output, transmit information, exchange information, capture measurements, and so on. The specific I/O components 1942 that are included in a particular machine will depend on the type of machine. For example, portable machines such as mobile phones will likely include a touch input device or other such input mechanisms, while a headless server machine will likely not include such a touch input device. It will be appreciated that the I/O components 1942 may include many other components that are not shown in FIG. 19. The I/O components 1942 are grouped according to functionality merely for simplifying the following discussion and the grouping is in no way limiting. In various example embodiments, the I/O components 1942 may include output components 1928 and input components 1930. The output components 1928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1942 may include biometric components 1932, motion components 1934, environmental components 1936, or position components 1938, among a wide array of other components. For example, the biometric components 1932 may include components to detect expressions (e.g., hand expressions, facial expressions, vocal expressions, body gestures, or eye tracking), measure biological signals (e.g., blood pressure, heart rate, body temperature, perspiration, or brain waves), identify a person (e.g., voice identification, retinal identification, facial identification, fingerprint identification, or electroencephalogram-based identification), and the like. The motion components 1934 may include acceleration sensor components (e.g., accelerometer), gravitation sensor components, rotation sensor components (e.g., gyroscope), and so forth. The environmental components 1936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1942 may include communication components 1940 operable to couple the machine 1900 to a network 1920 or devices 1922 via a coupling 1924 and a coupling 1926, respectively. For example, the communication components 1940 may include a network interface component or another suitable device to interface with the network 1920. In further examples, the communication components 1940 may include wired communication components, wireless communication components, cellular communication components, Near Field Communication (NFC) components, Bluetooth® components (e.g., Bluetooth® Low Energy), WiFi® components, and other communication components to provide communication via other modalities. The devices 1922 may be another machine or any of a wide variety of peripheral devices (e.g., a peripheral device coupled via a USB).

Moreover, the communication components 1940 may detect identifiers or include components operable to detect identifiers. For example, the communication components 1940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1940, such as location via Internet Protocol (IP) geolocation, location via Wi-Fi® signal triangulation, location via detecting an NFC beacon signal that may indicate a particular location, and so forth.

The various memories (i.e., memory 1904, main memory 1912, static memory 1914, and/or memory of the processors 1902) and/or storage unit 1916 may store one or more sets of instructions and data structures (e.g., software) embodying or utilized by any one or more of the methodologies or functions described herein. These instructions (e.g., the instructions 1908), when executed by processors 1902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1920 or a portion of the network 1920 may include a wireless or cellular network, and the coupling 1924 may be a Code Division Multiple Access (CDMA) connection, a Global System for Mobile communications (GSM) connection, or another type of cellular or wireless coupling. In this example, the coupling 1924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1908 may be transmitted or received over the network 1920 using a transmission medium via a network interface device (e.g., a network interface component included in the communication components 1940) and utilizing any one of several well-known transfer protocols (e.g., hypertext transfer protocol (HTTP)). Similarly, the instructions 1908 may be transmitted or received using a transmission medium via the coupling 1926 (e.g., a peer-to-peer coupling) to the devices 1922. The terms “transmission medium” and “signal medium” mean the same thing and may be used interchangeably in this disclosure. The terms “transmission medium” and “signal medium” shall be taken to include any intangible medium that can store, encoding, or carrying the instructions 1908 for execution by the machine 1900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

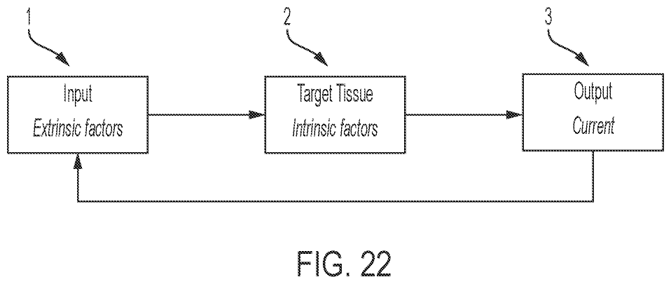

FIG. 22 depicts a diagram illustrating the relationship between an input 1, such as an extrinsic factor (as described herein), an intrinsic factor of the target tissue (as described herein), and the output 3 such as current and/or normalized current.

Furthermore, using the current normalization techniques and associated treatment planning tools described above, reversible electroporation procedures can be optimized by providing clinicians with information related to the onset of pore formation in cells, the extent of pore formation and the transition from reversible to irreversible zones. Reversible electroporation is typically used in a medical setting for mass transfer of chemical species (e.g., DNA, anti-cancer drugs, antibodies) into the cell interior, after which the cell regains hemostasis. Successful reversible electroporation techniques are dependent, in part, on the unique intrinsic and extrinsic characteristics of the treatment procedure, as discussed above. In addition, because substances are being introduced, the molecular size of the substance being introduced, number of pores being formed, the size of pore channels as well as the ability of the cell to recover after the application of electrical field are also factors affecting treatment outcomes.

FIG. 23 illustrates use of normalized current data to identify the onset of pore formation for reversible electroporation in zone zero and the transition to predominantly irreversible electroporation in zone one. In one example, zone zero represents the onset of pore formation for reversible electroporation that will occur within the first few pulses (i.e., the first 3-5 pulses). As the rate of normalized current increases after 3-5 pulses, zone zero (predominantly RE) transitions 2302 to zone one (predominantly IRE and/or HFIRE) which in this example may extend for at least 120 pulses. As show in FIG. 23, the slope of normalized current changes in steepness at around 20-21 pulses, with pulses 1-20 having a steeper slope for normalized current as compared with pulses deliver after pulse 21. This change in steepness in slope of normalized current is a result of the end of a round of pulses. After each round of pulses delivered to the target tissue, the conductivity (and reversibility of the pores in the cellular membrane) of the target tissue is changed. In one example, and as shown in FIG. 23, the first round of pulses ended at around pulse 20 and the second round of pulses started at around pulse 21. This change in round of pulses can be seen in the change in steepness of the slope of normalized current.

Banks of tissue-specific treatments from previously documented reversible electroporation procedures may also be used to optimize individual reversible electroporation treatment protocols. In addition to normalized current data, the bank may contain information on pore characteristics of specific cell types, including electrical field thresholds required to achieve onset of pore formation, the maximum electrical threshold before the cell type is unable to recover and pore size at a particular point in the procedure. The database may also include lookup tables on specific chemical species being introduced into the cell including but not limited to macromolecule type, size, and recommended pore size. As discussed above, the GUI interface may be used by the clinician to input treatment parameters specific to reversible electroporation such as macromolecule and target tissue cell type. Based on the clinician input and information from the databank, the therapy device may display recommended treatment parameters including recommended number of pulses to achieve optimal uptake by the cell while still maintaining cell viability. A benefit of the bank of tissue-specific treatment data includes, but is not limited to, predicting the current response after an increase or decrease in the applied voltage before or during a procedure and/or avoiding overcurrent. Clinicians may apply different voltages as treatment planning. They also may change the voltage during the procedure, which affect the current response. Therefore, by using a bank of tissue (of course normalized), you can predict the nature of current trend after the changed voltage point. This is important not only to predict the current trend after the changed voltage but also to avoid misunderstanding of current rise due to the ohmic effect and not IRE. Furthermore, by using the bank of normalized current for variety of tissue at different voltages, one will be able to predict the current trend, specially at the critical voltages where the chance of arcing is higher.

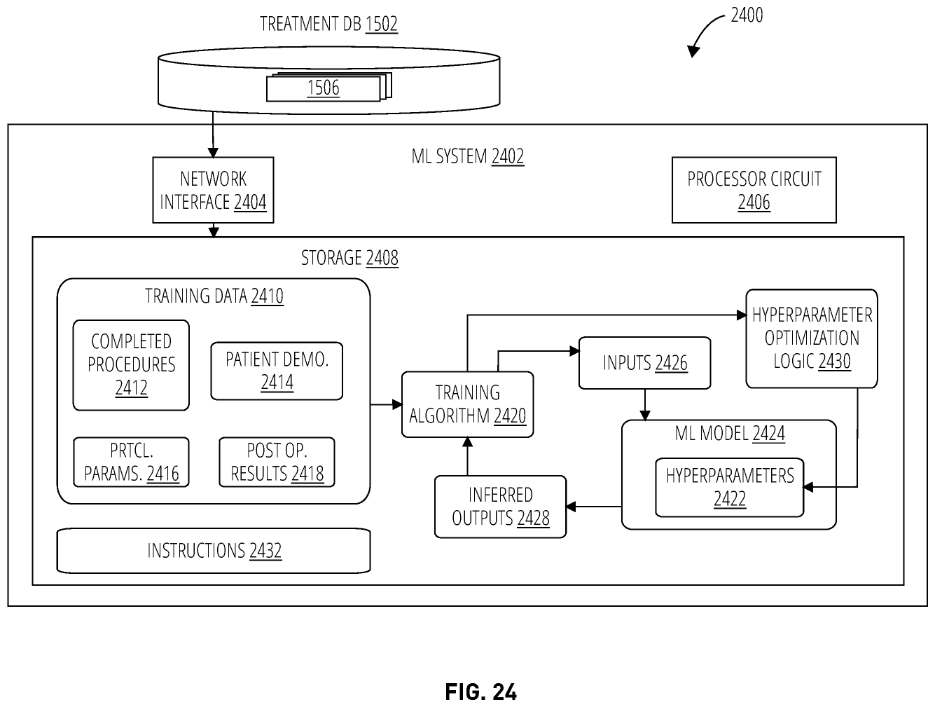

FIG. 24 illustrates a machine learning (ML) environment 2400, in accordance with non-limiting example(s) of the present disclosure. In general, ML environment 2400 can be implemented to apply ML to learn relationships between parameters of an IRE and/or H-FIRE treatment and results of the treatment to predict, or infer, parameters of the IRE and/or H-FIRE procedure. As a specific example, ML environment 2400 can be implemented to train an ML model to infer current levels, conductivity levels, protocol parameters, or other parameters of an IRE and/or H-FIRE procedure based on a number of inputs such as, voltage pulses applied during the IRE and/or H-FIRE procedure, extrinsic indications about the IRE and/or H-FIRE procedure, or the like.

A number of examples of training and using an ML model with an ablation therapy device (e.g., ablation therapy device 400, or the like) are provided herein while describing ML environment 2400. However, prior to providing details of ML environment 2400, it is noted that ML models are generally used in conjunction with an ablation therapy device to generate additional data points for a user (e.g., physician, technician, nurse, or the like) to use in managing a current or active ablation therapy procedure. IRE and/or H-FIRE ablation therapies are typically regarded as more complex than other treatment modalities (e.g., cryogenic therapies, thermal ablation therapies, radio frequency ablation therapies, etc.) by practicing physicians. For example, as described above, using conventional techniques it is difficult to accurately determine progress of treatments in real time, and more specifically when the application of additional electrical pulses causes the mechanism of cell death to change. Said differently, using conventional ablation therapy tools and data available via such tools, it is difficult for a physician to accurately determine when an ablation therapy transitions between therapy zones. Furthermore, it is not currently possible to compare different ablation therapies. That is, for two ablation therapies where the rounds of pulses were applied at different voltage amplitudes, comparing the transition between therapy zones of each individual therapy is not possible. These difficulties in both comparing ablation therapies and determining transitions between therapy zones lead to uncertainty. For example, these difficulties translate to difficulties for the physician to determine whether and when to adjust pulse parameters, whether to continue application of therapeutic pulses or whether to terminate pulse delivery.

The present disclosure provides to train and deploy ML models to generate an inference about an ablation therapy, which can aid a user (e.g., physician, technician, nurse, or the like) in pre-treatment planning, intra-treatment adjustment, and making determination of whether to continue and/or conclude delivery of therapeutic pulses during an ablation therapy. In particular, the present disclosure provides to use ML models combined with the normalized current techniques described above, which is described in greater detail herein.

The ML environment 2400 may include ML system 2402, such as a computing device that applies an ML algorithm to learn relationships between the above-noted items. The ML system 2402 may make use of treatment database 1502, which can be populated as described herein. With some examples, ML environment 2400 can be implemented as part of, or in conjunction with, ablation therapy system 1500. As a specific example, ML system 2402 could be implemented as part of ablation therapy device 400. However, for clarity, ML system 2402 is depicted and described as a separate device from ablation therapy device 400.

As described above, the treatment database 1502 may include information (e.g., patient data, pre-treatment data, treatment parameters, post-treatment data, etc.) collected during actual treatments, and from publicly available data, such as, from studies, registries done to support regulatory approvals, publications, electronic medical records, data repositories of individual medical treatment facilities, regional and national health centers, or the like. The treatment database 1502 may be remote from the ML system 2402 and accessed via a network interface 2404 (e.g., as depicted) or may be stored in a combination of local and remote data storage devices. For example, ML system 2402 may include a storage 2408, which may include a hard drive, solid state storage, and/or random access memory, which can store data associated with treatment database 1502 and treatment results 1506.

Storage 2408 stores training data 2410, which may comprise indications of IRE and/or H-FIRE completed procedures 2412 and patient demographics 2414 for the patient's undergoing the completed procedures 2412. Training data 2410 can also include indications of protocol parameters 2416 and post procedure results 2418 for the completed procedures 2412. As described in greater detail below, training data 2410 can be generated from data represented in treatment database 1502.

In general, protocol parameters 2416 can be representative of parameters related to planning the IRE and/or H-FIRE treatment. For example, protocol parameters 2416 can include indications of voltage amplitude, total number of voltage pulses, length of planned voltage pulses, information describing a train of voltage pulses, total on time, information describing a burst or bursts of voltage pulses, information describing cycles of voltage pulses, delay between voltage pulses, number of probes, probe type, spacing between probes, information describing a pattern or patterns of probe placement, probe polarity, relative to target (bracket vs. center), exposed length of the electrode(s), dimensions of the targeted ablation area, voltage/cm setting(s), model number of the voltage generator and/or ablation therapy device, software version of the voltage generator and/or ablation therapy device. As another example, protocol parameters 2416 can include indications about the IRE and/or H-FIRE procedure itself, such as, for example, cardiac sync, whether the procedure is open or closed, whether a paralytic is being used, indications of the initial conductivity of tissue (e.g., based on pre-treatment tests, or the like). With still other examples, protocol parameters 2416 could include indications of any number of the electric pulse parameters discussed above with respect to Table 2.

Patient demographics 2414 can include indications of the demographics for the patient undergoing the completed procedures 2412. For example, patient demographics 2414 can include indications of age, gender, race, insurance information, diagnosis, organ, cancer type, cancer stage, previous treatments, ongoing treatments (e.g., chemo therapies, focal therapies, or the like), co-morbidity scores, patient vitals (e.g., blood pressure, heart-rate, weight, height, or the like), location of cancer within organ, number of lesions, immune scores, imaging studies, implants, tumor location including non-tumor anatomical structures or proximate or in treatment zone (e.g., vessels, organs, bones, or the like), target tissue abnormalities (e.g., cysts, calcification, scar tissue, or the like).

Completed procedures 2412 can include data related to the actual procedure performed, such as, inter-procedure data and end of procedure data. For example, completed procedures 2412 can include indications of start and/or stop time of the procedure, the overall length of time of the procedure, data related to overcurrent conditions during the procedure (e.g., number of overcurrent conditions, amplitude of overcurrent conditions, or the like), adjustments made to any of the protocol parameters during the IRE and/or H-FIRE procedure, actual ones of the protocol parameters 2416 delivered during the IRE and/or H-FIRE procedure (e.g., total number of pulses delivered, number of pulses per probe pair, probe pair pulsing sequence, total pulse on time, current and voltage readings for each pulse delivered, or the like), probe repositioning info, intra or post procedure tissue information (e.g., resistance, conductivity readings, or the like), patient vitals during procedure, cardiac readings, procedural complications including mechanical damage due to probe insertion and/or adjustment, thermal heating, final ablation volumes and/or sizes, tissue conductivity changes not due to electrical pulses (e.g., saline flush, amount of intracellular fluids in target area, or the like). Furthermore, completed procedures 2312 can include indications of normalized current and/or normalized conductivity derived as outlined herein.

Post procedure results 2418 can include indications of information related to results of the procedure, such as, treatment complications (e.g., short term complications, long term complications, or the like), length of hospital stay, recovery times, survival rate (e.g., short term survival, long term survival, or the like), cancer recurrence, time to recurrence, disease free statistics, metastatic disease, quality of life measures, or the like.

Training data 2410 can be generated by ML system 2402. For example, processor circuit 2406 can execute instructions 2432 to generate training data 2410 from treatment results 1506 stored in treatment database 1502. In general, the training data 2410 may be applied to train ML model 2424. Depending on the particular application, different types of ML models 2424 may be suitable for use. For instance, in the depicted example, an artificial neural network (ANN) may be particularly well-suited to learning associations between completed procedures 2412, patient demographics 2414, protocol parameters 2416, and post procedure results 2418. Convoluted neural networks (CNNs) and random forest networks may also be well-suited to this particular type of task. However, one of ordinary skill in the art will recognize that different types of ML models 2424 may be used, depending on design goals, the resources available, the size of the dataset of training data 2410, etc.

Any suitable training algorithm 2420 may be used to train the ML model 2424. Nonetheless, the example depicted in FIG. 24 may be particularly well-suited to a supervised training algorithm or reinforcement learning. For a supervised training algorithm, the ML system 2402 may apply inputs 2426 to the ML model 2424 while the model generates an inference, such as, inferred outputs 2428 based on the inputs. In some examples the completed procedures 2412, patient demographics 2414, and post procedure results 2418 can be applied as inputs 2426 to map these portions of training data to protocol parameters 2416. It is to be appreciated that the aim of “training” the ML model 2424 is for the ML model 2424 to learn associations between the inputs 2426 (e.g., completed procedures 2412, patient demographics 2414, and post procedure results 2418) and inferred outputs 2428 (e.g., protocol parameters 2416).

ML models (e.g., ML model 2424) have hyperparameters 2422. Hyperparameters 2422 can include a variety of items related to the ML model 2424, such as, for example, number of nodes, number of layers, number of hidden layers, value of weights connecting each node, the activation function of each node, the learning gradient, etc. In a reinforcement learning scenario, hyperparameters 2422 of the ML model 2424 are adjusted, based on the training algorithm 2420 with the goal being that the inferred outputs 2428 converge upon an acceptable level of accuracy to what the inferred outputs 2428 are expected to be.

The training algorithm 2420 may be applied using processor circuit 2406, which may include suitable hardware processing resources that operate on the logic and structures in the storage 2408. As noted, training algorithm 2420 and/or the development of the trained ML model 2424 is at least partially dependent on model hyperparameters 2422. In exemplary examples, the model hyperparameters 2422 can be automatically selected based on hyperparameter optimization logic 2430, which may include any known hyperparameter optimization techniques as appropriate to the ML model 2424 selected and the training algorithm 2420 to be used.

In some embodiments, some of the training data 2410 may be used to initially train the ML model 2424 while some of the training data 2410 can be reserved and used as a validation subset. The portion of the training data 2410 not including the validation subset may be used to train the ML model 2424 whereas the validation subset may be used to test the trained ML model 2424 and to verify that the ML model 2424 is able to generalize or correctly infer outputs from unseen or new data.

In optional examples, the ML model 2424 may be re-trained over time, for example, to accommodate knowledge about updated, new, recent, or otherwise different procedures and associated protocol parameters not reflected in the training data 2410 with which the ML model 2424 was previously trained on. As a specific example, ML model 2424 can be repeatedly (e.g., on a fixed period, as sufficient new data exists, or the like) trained to account for various updates in the data set (e.g., updates in physician preferences, updates in accepted treatment guidelines, new academic research, new clinical trials or studies, or the like). Along these lines, with many examples, treatment database 1502 can be expanded over time. Furthermore, with some examples, portions of treatment database 1502 (e.g., completed procedures 2412, patient demographics 2414, protocol parameters 2416, or the like) can be populated around the time of an IRE and/or H-FIRE procedure while other portions (e.g., post procedure results 2418, or the like) of treatment database 1502 can be populated subsequent to the procedure, possibly by a different user (e.g., different physician, different technician, different nurse, or the like). As such, an updated version of training data 2410 can be generated from an expanded treatment database 1502.

Once the ML model 2424 is trained, it may be executed, for example, by processor circuit 2406 (or another processor circuit, such as, processor 408 of ablation therapy device 400) to new input data. As a specific example, ML model 2424 can be executed by processing circuitry of an ablation therapy device to generate an inference about protocol parameters 2416 from inputs related to a current IRE and/or H-FIRE procedure for which the ablation therapy device is to be used. This input to the ML model 2424 may be formatted according to a predefined format, which for example, can mirror the way that the training data 2410 was provided to the ML model 2424. The ML model 2424 may generate inferred outputs 2428 which may be, for example, a prediction of normalized currents, tissue conductivities, protocol parameters, or the like based on the provided inputs.

The inferred outputs 2428 may be provided to a user of the ablation therapy device (e.g., physician, nurse, technician, or the like) as a recommendation for protocol parameters to select for a current IRE and/or H-FIRE procedure or as another data point to use in adjusting and/or concluding the procedure.

The above description pertains to a particular kind of ML system 2402, which applies supervised learning techniques given available training data with input/output pairings. However, the present disclosure is not limited to use with a specific ML paradigm, and other types of ML techniques may be used. For example, in some embodiments the ML system 2402 may apply other types of ML techniques, such as evolutionary algorithms, without departing from the scope of the disclosure.

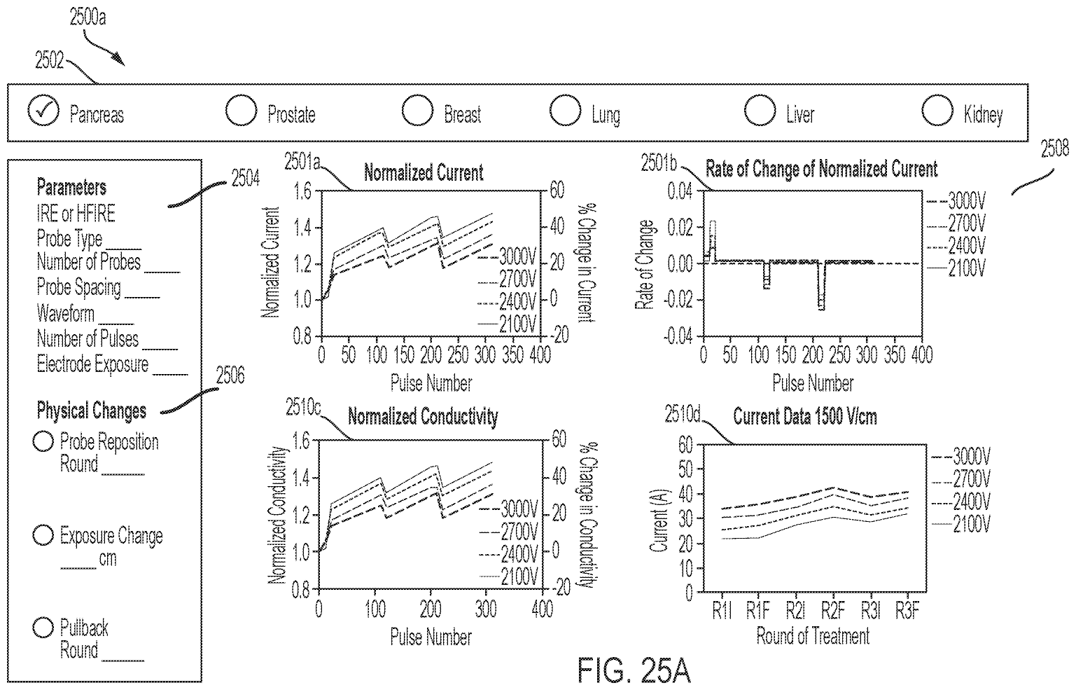

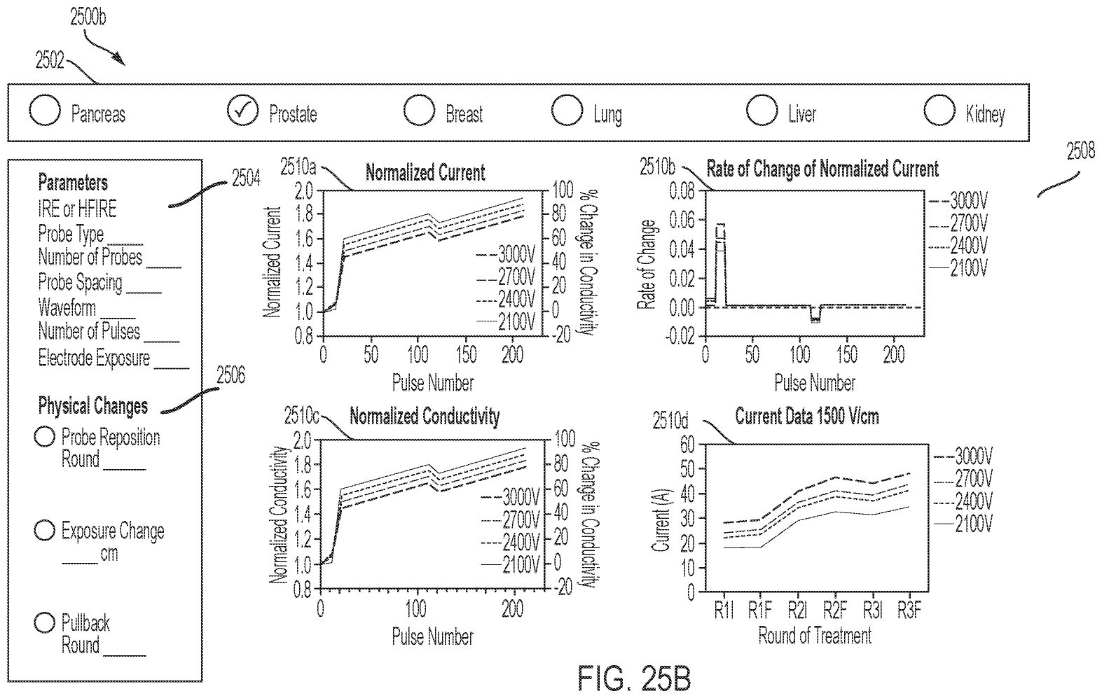

FIG. 25A and FIG. 25B illustrates graphical display 2500a and graphical display 2500b, respectively, which can be generated by an ablation therapy device (e.g., ablation therapy device 400, or the like) and presented on a display for a clinician to use in pre-planning, intra-treatment adjustment, and conclusion of an IRE and/or H-FIRE procedure. For example, ablation therapy device 400 can generate graphical display 2500a and/or graphical display 2500b and present on display 410. As a specific example, processor 408 in executing instructions 416 can generate display data (e.g., display frames, or the like) comprising the graphical elements represented in these figures.

An ablation therapy device (e.g., ablation therapy device 400, or the like) can generate graphical display 2500a or graphical display 2500b comprising an indication of a tissue type selector 2502, a parameters selector 2504, an intra-treatment adjustments selector 2506, and a treatment characteristics 2508. Furthermore, ablation therapy device 400 can be configured, as described above, to receive an indication of a tissue type (e.g., from a physician, or the like) and generate a graphical display comprising the received tissue type. For example, FIG. 25A depicts displaying an indication of a selected tissue type pancreas in tissue type selector 2502 while FIG. 25B depicts displaying an indication of a selected tissue type prostate in tissue type selector 2502. It is noted that the available tissue types for selection can be depended upon the tissue types represented in treatment database 1502 or the tissue types with which ML model 2424 is trained and that the tissue type examples depicted in these figures is given for example only.

The ablation therapy device 400 can further be configured, as described above, to receive an indication of treatment parameters (e.g., IRE or H-FIRE, probe type, number of probes, probe spacing, waveform parameters, number of pulses, electrode exposure length, treatment zone size, margin size, or the like) from a user (e.g., physician, or the like). Ablation therapy device 400 can generate graphical display 2500a and/or graphical display 2500b comprising the treatment parameters selected by the user via parameters selector 2504.

Ablation therapy device 400 can generate graphical display 2500a and/or graphical display 2500b including treatment characteristics 2508. As depicted, treatment characteristics 2508 comprises a number of plots associated with an ablation therapy treatment having the parameters indicated in 2504 for the type of tissue reflected in tissue type selector 2502. With some examples, data (e.g., plots, or the like) depicted in treatment characteristics 2508 can be generated by ML model 2424 based on input from tissue type selector 2502 and parameters selector 2504. For example, ML model 2424 can generate plots plot 2510a, 2510b, 2510c, and 2510d as output based on the described inputs. As a specific example, as depicted in these figures, plots 2510a, 2510b, 2510c, and 2510d can be generated (e.g., by ML model 2424) comprising an indication of normalized current versus pulse number (e.g., plot 2510a), normalized tissue conductivity versus pulse number (e.g., plot 2510b), a rate of change of normalized current versus pulse number (e.g., plot 2510c), and a voltage gradient (V/cm) versus a round (number of pulses) of treatment (e.g., plot 2510d). It is noted that the depicted plots are given for example only and different plots or more or less plots than the depicted can be generated and displayed in graphical display 2500a and/or graphical display 2500b.

During an active treatment, ablation therapy device 400 can repeatedly (e.g., on a fixed period, after a number of pulses, after voltage or current level thresholds, or the like) update treatment characteristics 2508. For example, ML model 2424 can be arranged to generate updated plots, such as, updated versions of plots 2510a, 2510b, 2510c, and 2510d based on intra-treatment measurements. For example, ML model 2424 can be configured to generate updated plots based on intra-treatment extrinsic measurements (e.g., current, voltage, or the like). Furthermore, ablation therapy device 400 can further be configured, as described above, to receive an indication of changes to be made intra-treatment from a user (e.g., physician, or the like). As an example, ablation therapy device 400 can be configured to receive, intra-treatment, indications to stop the procedure, stop delivery of therapeutic pulses for one or multiple probe pairs, deactivate one or multiple probe pairs, reactivate one or multiple probe pairs, reactivate delivery of therapeutic pulses, adjust electrocardiogram (ECG) leads, account for repositioned probes, account for changed electrode exposure length, adjust therapeutic pulse parameters (e.g., voltage amplitude, voltage/cm, total number of pulses, pulse width, maximum allowable current and/or conductivity, intrapulse delay, polarity of pulses, delay between sequences or trains of pulses, or the like).

Ablation therapy device 400 can generate graphical display 2500a and/or graphical display 2500b comprising the intra-treatment changes via intra-treatment adjustments selector 2506. Furthermore, ablation therapy device 400 can update treatment characteristics 2508 based on the changes reflected in intra-treatment adjustments selector 2506. For example, ML model 2424 can be arranged to generate updated plots, such as, updated versions of plots 2510a, 2510b, 2510c, and 2510d based on inputs from tissue type selector 2502, parameters selector 2504, intra-treatment adjustments selector 2506, and/or other extrinsic data related to the active ablation therapy.

As noted above, the complexity of ablation therapies often leads to difficulty in a therapy provider (e.g., clinician, or the like) making a determination of how to adjust a therapy intra-treatment as well as when to conclude a therapy. The present disclosure provides more than merely collecting, analyzing, and displaying information related to an ablation therapy. Instead, the present disclosure provides a unique system wherein data from different types of IRE and/or H-FIRE treatments having different protocols (intrinsic and extrinsic values) can be compared, such as, via normalized current. Given the details provided herein regarding normalized current, ML model 2424 can be trained on many different ablation therapies (e.g., a reflected in treatment database 1502) even where these ablation therapies used different parameters. Accordingly, information about therapy zones for a current ablation therapy can be generated where such information is not available conventionally. Said differently, the information generated herein and displayed in treatment characteristics 2508 is not information that is conventionally available to collect and analyze. Furthermore, as provided herein ML model 2324 can be trained to generalize normalized currents from any combination of input parameters based on normalized currents for prior therapies with outcomes meeting selected criteria (e.g., as reflected in treatment database 1502). It is emphasized that this is not conventionally possible and is significantly more than merely collecting, analyzing, and displaying information.

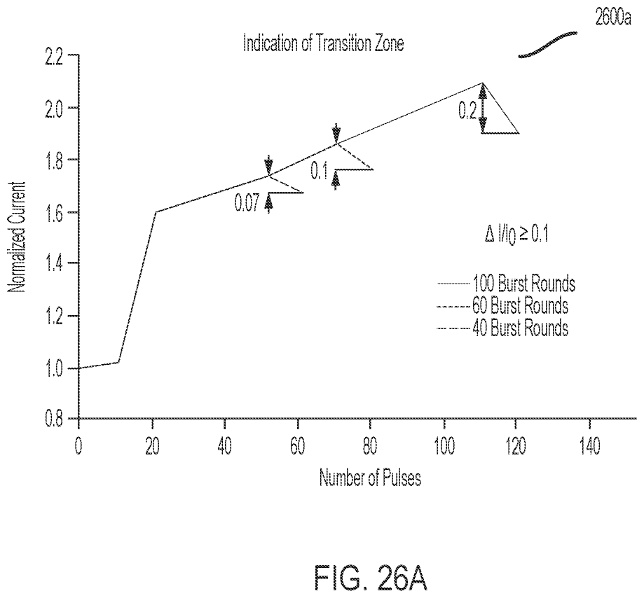

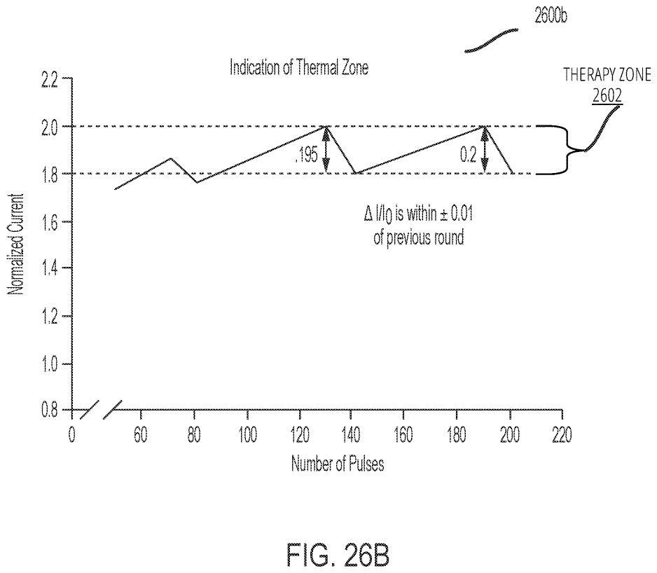

As noted, in some examples, treatment characteristics 2508 can include more or less plots than depicted in graphical display 2500a and graphical display 2500b of FIG. 25A and FIG. 25B. For example, FIG. 26A and FIG. 26B depict examples of plots that can be generated by ML model 2424 from inputs reflected in tissue type selector 2502, parameters selector 2504, intra-treatment adjustments selector 2506, and/or other extrinsic data. A graphical display (e.g., graphical display 2500a and/or graphical display 2500b) can be generated to include indications of these plots. Said differently, ML model 2424 can be trained to infer normalized current for an ablation therapy to account for extrinsic and/or intrinsic characteristics of the ablation therapy. Ablation therapy device 400 can generate, based on the inference of normalized current from ML model 2424, plots representing drops in normalized current (e.g., per pulse, per rounds of pulses, or the like) to aid a user in conducting the ablation therapy, such as with pre-treatment planning, intra-treatment adjustments, and treatment conclusion determinations.

FIG. 26A depicts plot 2600a. Plot 2600a is a representation of normalized current for a number of pulses where indications of transition between therapy zones for different number of pulses per round is depicted. Specifically, plot 2600a depicts normalized current and where a transition between therapy zones will occur for 40 burst rounds, 60 burst rounds, and 100 burst rounds. With some examples, ML model 2424 can be configured to generate plot 2600a, as described above, and ablation therapy device 400 can generate treatment characteristics 2508 including an indication of plot 2600a.

As a specific example, ablation therapy device 400 can be arranged to generate (e.g., based on inferences of ML model 2424) a depiction including the plot 2600a. A user (e.g., a physician, or the like) may use the information provided in plot 2600a to determine parameters to use for an ablation therapy with the ablation therapy device 400. In some therapies, transition between therapy zones is indicated by a drop in normalized current of greater than or equal to 0.1. Accordingly, if the user wanted to cause a transition between therapy zones (e.g., between zone 1 and zone 2, or the like) the user could select parameters with which plot 2600a indicates would cause a drop in normalized current of greater than or equal 0.1. Specifically, a user might select multiple rounds of 40 pulses (e.g., 40 burst rounds) or a single round of 60 pulses or 100 pulses (e.g., a 60 burst round or a 100 burst round) to achieve the desired drop in normalized current of greater than or equal to 0.1. In some examples, a user may elect to apply multiple (e.g., 2, etc.) rounds of 40 pulses to potentially reduce a thermal rise in the tissue due to the delay between rounds. However, another user may elect to apply a single round of 60 or 100 pulses in order to transition between the therapy zones more quickly.

FIG. 26B depicts plot 2600b. Plot 2600b is a representation of normalized current for a number of pulses where, based on the number of pulses, an ablation therapy may exit the IRE therapy zone. Specifically, plot 2600b depicts normalized current for a number of potential pulses and where a transition 2602 out of a therapy zone may be, based on the normalized current. With some examples, ML model 2424 can be configured to generate plot 2600b, as described above, and ablation therapy device 400 can generate treatment characteristics 2508 including an indication of plot 2600b. Said differently, ML model 2424 can be arranged to infer normalized current and ablation therapy device 400 can be arranged to generate a depiction including the plot 2600a from the inferred normalized current. A user (e.g., a physician, or the like) may use the information provided in plot 2600b to determine when to conclude an ablation therapy conducted with the ablation therapy device 400. In some therapies, transition out of a therapy zone is indicated by a drop in normalized current after a round of pulses of less than or equal to 0.01 of the drop in normalized current after the previous round of pulses. For example, transition 2602 shows a drop in normalized current of less than or equal to 0.01 the previous drop in normalized current. Accordingly, if the user wanted to conclude an ablation therapy at the transition 2602, the user could elect to stop the ablation therapy after the number of pulses indicated in plot 2600b corresponding to the transition 2602.

What is claimed is:

1. An ablation therapy device, comprising:
a generator, a sensor, a processor, and a memory, the processor coupled to the generator, the sensor, and the memory; the generator to operatively couple to a plurality of electrodes, and the generator to generate a plurality of electrical pulses to be applied through the electrodes to a target tissue;
the sensor arranged to measure a current produced responsive to application of the plurality of electrical pulses to the target tissue; and
memory storing instructions, which when executed by the processor cause the processor to:
receive from the sensor, an indication of the current; and
normalize the current.

2. The device of claim 1, the instructions, when executed by the processor cause the processor to:
determine whether a difference between the normalized current for a first electrical pulse of the plurality of electrical pulses and the normalized current for a second electrical pulse of the plurality of electrical pulses is greater than a threshold value; and
generate a control signal comprising an indication to pause generation of the plurality of electrical pulses based on a determination that the difference between the normalized current for the first electrical pulse of the plurality of electrical pulses and the normalized current for the second electrical pulse of the plurality of electrical pulses is greater than the threshold value.

3. The device of claim 1, further comprising a display unit coupled to the processor; and the instructions, when executed by the processor cause the processor to:
generate a first graphical information element comprising an indication of a plot of the normalized current;
generate a second graphical information element comprising an indication of a query of whether to continue generation of the plurality of electrical pulses; and
send the first graphical information element and the second graphical information element to the display unit to cause the display unit to display the plot and the query.

4. The device of claim 1, wherein the sensor comprises a voltage sensor, a current sensor, or a voltage sensor and a current sensor.

5. The device of claim 1, wherein the plurality of electrical pulses are sufficient to substantially reversibly electroporate cells within the target tissue, irreversibly electroporate cells within the target tissue, thermally ablate cells within the target tissue, and/or result in electrolysis of cells within the target tissue.

6. The device of claim 1, wherein the normalized current comprises an extrinsic factors and an intrinsic factors.

7. The device of claim 1, the instructions, when executed by the processor cause the processor to normalize a rate of change of the current.

8. The device of claim 1, wherein the sensor arranged to measure a conductivity produced responsive to the application of the plurality of electrical pulses to the target tissue; and wherein the memory storing instructions, which when executed by the processor cause the processor to:
receive from the sensor, an indication of the conductivity; and
normalize the conductivity.

9. The device of claim 1, the instructions, when executed by the processor cause the processor to:
generate a control signal based on the normalized current; and
send the control signal to the generator.

10. The device of claim 1, further comprising a memory storing a machine learning (ML) model and instructions, the instructions when executed by the processor cause the processor to:
execute the ML model to generate an inference of the normalized current based on the indication of the sensed current.

11. An ablation device, comprising:
a voltage source to generate a plurality of electrical pulses to be applied to a target site, the voltage source to operatively couple to a plurality of electrodes;
a sensor coupled to at least one of the plurality of electrodes, the sensor arranged to measure an electrical characteristic associated with application of the plurality of electrical pulses to the target tissue;
a processor; and
memory storing a machine learning (ML) model and instructions, the instructions when executed by the processor cause the processor to:
receive from the sensor, an indication of the electrical characteristic;
normalize the electrical characteristic;
execute the ML model to generate an inference of the normalized electrical characteristic based on the indication of the electrical characteristic; and
generate a graphical information element comprising the indication of the normalized electrical characteristic of the ablation therapy.

12. The device of claim 11, wherein the sensor comprises a voltage sensor, a current sensor, or a voltage sensor and a current sensor; wherein the electrical characteristic comprises a current, a voltage, or a current and a voltage; and wherein the normalized electrical characteristic comprises intrinsic factors and/or extrinsic factors.

13. The device of claim 11, the instructions, when executed by the processor cause the processor to:
receive an indication of a type of the target tissue; and
execute the ML model to generate the inference of the normalized electrical characteristic of the ablation therapy based on the indication of the normalized electrical characteristic and the type of the target tissue.

14. The ablation therapy device of claim 13, the instructions, when executed by the processor cause the processor to:
receive at least one of an indication of patient demographics or an indication of protocol parameters of the ablation therapy; and
execute the ML model to generate the inference of the normalized electrical characteristic of the ablation therapy based on the indication of the normalized electrical characteristic, the type of the target tissue, and the at least one of the indications of patient demographics or the indication of protocol parameters of the ablation therapy.

15. The ablation therapy device of claim 14, wherein the normalized electrical characteristic of the ablation therapy comprises one or more of normalized current, normalized conductivity, an ablation therapy zone, a rate of change in normalized current versus a quantity of the plurality of voltage pulses, or a rate of change in normalized conductivity versus the quantity of the plurality of voltage pulses.

16. The ablation therapy device of claim 11, the instructions, when executed by the processor cause the processor to:
receive an indication from the voltage source of a second plurality of voltage pulses to be applied to the target tissue via the plurality of electrodes as part of the ablation therapy;
execute the ML model to generate an updated inference of an updated normalized electrical characteristic of the ablation therapy based on the electrical characteristic and the second plurality of voltage pulses; and
generate a second graphical information element comprising the indication of the updated normalized electrical characteristic of the ablation therapy.

17. The ablation therapy device of claim 11, the instructions when executed by the processor cause the processor to:
execute the ML model to generate an inference of the normalized electrical characteristic and suggested protocol parameters of the ablation therapy; and
generate the graphical information element comprising the indication of the normalized electrical characteristic of the ablation therapy and an indication of the suggested protocol parameters.

18. A method, comprising:
receiving from a sensor, an indication of an electrical characteristic generated responsive to at least one electrical pulse applied to a target tissue by a plurality of electrodes operatively coupled to an ablation therapy device;
normalizing the electrical characteristic;
generating a control signal for the ablation therapy device based on the normalized electrical characteristic; and
sending the control signal to the ablation therapy device.

19. The method of claim 18, wherein the step of normalizing the electrical characteristic comprises both extrinsic factors and intrinsic factors.

20. The method of claim 18, wherein the sensor comprises a voltage sensor, a current sensor, or a voltage sensor and a current sensor and wherein the electrical characteristic comprises a current, a voltage, or a current and a voltage; and further comprising the steps:
generating a first graphical information element comprising an indication of a plot of the normalized electrical characteristic;
generating a second graphical information element comprising an indication of a query of whether to continue application of the plurality of electrical pulses; and
displaying on a display device, based on the first graphical information element and the second graphical information element, the plot and the query.
